SEQUENCE LISTING (93 sequences)

SEQ ID NO: 1 - protein, 214 aa, Artificial sequence
  Translation of common light chain (cLC)
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 7 aa, Homo sapiens
ESKYGPP

SEQ ID NO: 3 - protein, 10 aa, Homo sapiens
EPKSCDKTHT

SEQ ID NO: 4 - protein, 10 aa, Artificial sequence
  Linker
GGGGSGGGGS

SEQ ID NO: 5 - protein, 12 aa, Artificial sequence
  Linker
ERKSSVESPPSP

SEQ ID NO: 6 - protein, 12 aa, Artificial sequence
  Linker
ERKCSVESPPSP

SEQ ID NO: 7 - protein, 12 aa, Homo sapiens
ELKTPLGDTTHT

SEQ ID NO: 8 - protein, 12 aa, Artificial sequence
  Linker
ESKYGPPSPSSP

SEQ ID NO: 9 - protein, 13 aa, Artificial sequence
  Linker
ERKSSVEAPPVAG

SEQ ID NO: 10 - protein, 13 aa, Artificial sequence
  Linker
ERKCSVEAPPVAG

SEQ ID NO: 11 - protein, 14 aa, Artificial sequence
  Linker
ESKYGPPAPEFLGG

SEQ ID NO: 12 - protein, 15 aa, Artificial sequence
  Linker
EPKSCDKTHTSPPSP

SEQ ID NO: 13 - protein, 16 aa, Artificial sequence
  Linker
EPKSCDGGGGSGGGGS

SEQ ID NO: 14 - protein, 16 aa, Artificial sequence
  Linker
GGGGSGGGGSAPPVAG

SEQ ID NO: 15 - protein, 17 aa, Artificial sequence
  Linker
EPKSCDKTHTAPELLGG

SEQ ID NO: 16 - protein, 18 aa, Artificial sequence
  Linker
ERKSSVESPPSPAPPVAG

SEQ ID NO: 17 - protein, 18 aa, Artificial sequence
  Linker
ERKCSVESPPSPAPPVAG

SEQ ID NO: 18 - protein, 19 aa, Artificial sequence
  Linker
ELKTPLGDTTHTAPEFLGG

SEQ ID NO: 19 - protein, 19 aa, Artificial sequence
  Linker
ESKYGPPSPSSPAPEFLGG

SEQ ID NO: 20 - protein, 22 aa, Artificial sequence
  Linker
EPKSCDKTHTSPPSPAPELLGG

SEQ ID NO: 21 - protein, 23 aa, Artificial sequence
  Linker
ERKSSVEEAAAKEAAAKAPPVAG

SEQ ID NO: 22 - protein, 23 aa, Artificial sequence
  Linker
ERKCSVEEAAAKEAAAKAPPVAG

SEQ ID NO: 23 - protein, 24 aa, Artificial sequence
  Linker
ESKYGPPEAAAKEAAAKAPEFLGG

SEQ ID NO: 24 - protein, 27 aa, Artificial sequence
  Linker
EPKSCDKTHTEAAAKEAAAKAPELLGG

SEQ ID NO: 25 - protein, 29 aa, Artificial sequence
  Linker
ELKTPLGDTTHTEAAAKEAAAKAPEFLGG

SEQ ID NO: 26 - protein, 22 aa, Homo sapiens
EPKSCDKTHTCPPCPAPELLGG

SEQ ID NO: 27 - protein, 5 aa, Homo sapiens
CPPCP

SEQ ID NO: 28 - protein, 7 aa, Homo sapiens
APELLGG

SEQ ID NO: 29 - protein, 18 aa, Homo sapiens
ERKCCVECPPCPAPPVAG

SEQ ID NO: 30 - protein, 7 aa, Homo sapiens
ERKCCVE

SEQ ID NO: 31 - protein, 6 aa, Homo sapiens
APPVAG

SEQ ID NO: 32 - protein, 69 aa, Homo sapiens
ELKTPLGDTTHTCPRCPEPKSCDTPPPCPRCPEPKSCDTPPPCPRCPEPKSCDTPPPCPRCPAPEFLGG

SEQ ID NO: 33 - protein, 50 aa, Homo sapiens
CPRCPEPKSCDTPPPCPRCPEPKSCDTPPPCPRCPEPKSCDTPPPCPRCP

SEQ ID NO: 34 - protein, 7 aa, Homo sapiens
APEFLGG

SEQ ID NO: 35 - protein, 19 aa, Homo sapiens
ESKYGPPCPSCPAPEFLGG

SEQ ID NO: 36 - protein, 5 aa, Homo sapiens
CPSCP

SEQ ID NO: 37 - protein, 52 aa, Artificial sequence
  Synthetic construct
NVNHKPSNTKVDKRVEPKSCDKTHTSPPSPAPELLGGEVQLVESGGGVVQPG

SEQ ID NO: 38 - protein, 45 aa, Artificial sequence
  NVNHKPSNTKVDKRVEPKSCDKTHTSPPSPEVQLVESGGGVVQPG
NVNHKPSNTKVDKRVEPKSCDKTHTSPPSPEVQLVESGGGVVQPG

SEQ ID NO: 39 - protein, 40 aa, Artificial sequence
  Synthetic construct
NVNHKPSNTKVDKRVEPKSCDKTHTEVQLVESGGGVVQPG

SEQ ID NO: 40 - protein, 46 aa, Artificial sequence
  Synthetic construct
NVNHKPSNTKVDKRVEPKSCDGGGGSGGGGSEVQLVESGGGVVQPG

SEQ ID NO: 41 - protein, 57 aa, Artificial sequence
  Synthetic construct
NVNHKPSNTKVDKRVEPKSCDKTHTEAAAKEAAAKAPELLGGEVQLVESGGGVVQPG

SEQ ID NO: 42 - protein, 47 aa, Artificial sequence
  Synthetic construct
NVNHKPSNTKVDKRVEPKSCDKTHTAPELLGGEVQLVESGGGVVQPG

SEQ ID NO: 43 - protein, 48 aa, Artificial sequence
  Synthetic construct
NVDHKPSNTKVDKTVERKSSVESPPSPAPPVAGEVQLVESGGGVVQPG

SEQ ID NO: 44 - protein, 42 aa, Artificial sequence
  Synthetic construct
NVDHKPSNTKVDKTVERKSSVESPPSPEVQLVESGGGVVQPG

SEQ ID NO: 45 - protein, 43 aa, Artificial sequence
  Synthetic construct
NVDHKPSNTKVDKTVERKSSVEAPPVAGEVQLVESGGGVVQPG

SEQ ID NO: 46 - protein, 48 aa, Artificial sequence
  Synthetic construct
NVDHKPSNTKVDKTVERKCSVESPPSPAPPVAGEVQLVESGGGVVQPG

SEQ ID NO: 47 - protein, 42 aa, Artificial sequence
  Synthetic construct
NVDHKPSNTKVDKTVERKCSVESPPSPEVQLVESGGGVVQPG

SEQ ID NO: 48 - protein, 43 aa, Artificial sequence
  Synthetic construct
NVDHKPSNTKVDKTVERKCSVEAPPVAGEVQLVESGGGVVQPG

SEQ ID NO: 49 - protein, 46 aa, Artificial sequence
  Synthetic construct
NVDHKPSNTKVDKTVGGGGSGGGGSAPPVAGEVQLVESGGGVVQPG

SEQ ID NO: 50 - protein, 40 aa, Artificial sequence
  Synthetic construct
NVDHKPSNTKVDKTVGGGGSGGGGSEVQLVESGGGVVQPG

SEQ ID NO: 51 - protein, 53 aa, Artificial sequence
  Synthetic construct
NVDHKPSNTKVDKTVERKSSVEEAAAKEAAAKAPPVAGEVQLVESGGGVVQPG

SEQ ID NO: 52 - protein, 53 aa, Artificial sequence
  Synthetic construct
NVDHKPSNTKVDKTVERKCSVEEAAAKEAAAKAPPVAGEVQLVESGGGVVQPG

SEQ ID NO: 53 - protein, 49 aa, Artificial sequence
  Synthetic construct
NVNHKPSNTKVDKRVELKTPLGDTTHTAPEFLGGEVQLVESGGGVVQPG

SEQ ID NO: 54 - protein, 42 aa, Artificial sequence
  Synthetic construct
NVNHKPSNTKVDKRVELKTPLGDTTHTEVQLVESGGGVVQPG

SEQ ID NO: 55 - protein, 59 aa, Artificial sequence
  Synthetic construct
NVNHKPSNTKVDKRVELKTPLGDTTHTEAAAKEAAAKAPEFLGGEVQLVESGGGVVQPG

SEQ ID NO: 56 - protein, 49 aa, Artificial sequence
  Synthetic construct
NVDHKPSNTKVDKRVESKYGPPSPSSPAPEFLGGEVQLVESGGGVVQPG

SEQ ID NO: 57 - protein, 42 aa, Artificial sequence
  Synthetic construct
NVDHKPSNTKVDKRVESKYGPPSPSSPEVQLVESGGGVVQPG

SEQ ID NO: 58 - protein, 44 aa, Artificial sequence
  Synthetic construct
NVDHKPSNTKVDKRVESKYGPPAPEFLGGEVQLVESGGGVVQPG

SEQ ID NO: 59 - protein, 37 aa, Artificial sequence
  Synthetic construct
NVDHKPSNTKVDKRVESKYGPPEVQLVESGGGVVQPG

SEQ ID NO: 60 - protein, 54 aa, Artificial sequence
  Synthetic construct
NVDHKPSNTKVDKRVESKYGPPEAAAKEAAAKAPEFLGGEVQLVESGGGVVQPG

SEQ ID NO: 61 - protein, 52 aa, Artificial sequence
  Synthetic construct
NVNHKPSNTKVDKRVEPKSCDKTHTCPPCPAPELLGGPSVFLFPPKPKDTLM

SEQ ID NO: 62 - DNA, 321 bp, Artificial sequence
  common light chain variable domain (IGKV1-39/jk1)
  CDS (1)..(321)
gacatccagatgacccagtctccatcctccctgtctgcatctgtaggagacagagtcaccatcacttgccgggcaagtcagagcattagcagctacttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccaacgttcggccaagggaccaaggtggagatcaaa

SEQ ID NO: 63 - protein, 107 aa, Artificial sequence
  Synthetic Construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 64 - protein, 107 aa, Artificial sequence [sequence omitted]

SEQ ID NO: 65 - protein, 95 aa, Artificial sequence
  V-region IGKV1-39A
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 66 - DNA, 382 bp, Artificial Sequence
  MF6058 in Mase DNA
ggcccagccggccatggcccaggtgcagctggtgcagtctggggctgacgtgaagaagcctggggcctcagtgaaggtcacgtgcaaggcttctggatacaccttcaccggctactatatgcactgggtgcgacaggcccctggacaagctcttgagtggatgggatggatcaaccctcaaagtggtggcacaaactatgcaaagaagtttcagggcagggtctctatgaccagggagacgtccacaagcacagcctacatgcagctgagcaggctgagatctgacgacacggctacgtattactgtgcaagagatcatggttctcgtcatttctggtcttactggggctttgattattggggccaaggtaccctggtcacc

SEQ ID NO: 67 - protein, 121 aa, Artificial Sequence
  MF6058 in Mase AA
QVQLVQSGADVKKPGASVKVTCKASGYTFTGYYMHWVRQAPGQALEWMGWINPQSGGTNYAKKFQGRVSMTRETSTSTAYMQLSRLRSDDTATYYCARDHGSRHFWSYWGFDYWGQGTLVT

SEQ ID NO: 68 - DNA, 354 bp, Artificial Sequence
  MF3004 DNA
caggtgcagctgaagcagtctggggctgagctggtgaggcctggggcttcagtgaagctgtcctgcaaggcttctggctacactttcactggctactatataaactgggtgaagcagaggcctggacagggacttgagtggattgcaaggatttatcctggaagtggttatacttactacaatgagaagttcaagggcaaggccacactgactgcagaagaatcctccagcactgcctacatgcacctcagcagcctgacatctgaggactctgctgtctatttctgtgcaagaccccactatggttacgacgactggtacttcggtgtctggggcacaggcaccacggtcacc

SEQ ID NO: 69 - protein, 118 aa, Artificial Sequence
  MF3004 AA
QVQLKQSGAELVRPGASVKLSCKASGYTFTGYYINWVKQRPGQGLEWIARIYPGSGYTYYNEKFKGKATLTAEESSSTAYMHLSSLTSEDSAVYFCARPHYGYDDWYFGVWGTGTTVT

SEQ ID NO: 70 - DNA, 372 bp, Artificial Sequence
  MF1337 DNA
gaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcacc

SEQ ID NO: 71 - protein, 124 aa, Artificial Sequence
  MF1337 AA
EVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVT

SEQ ID NO: 72 - DNA, 1099 bp, Artificial Sequence
  DNA encoding MF1337-CH1-IgG1 G4S linker-MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggcccatcggtcttccccctggcaccctcctccaagtccacgtctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaagagctgcgacggcggtggcggcagcggcggcggaggcagcgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 73 - protein, 366 aa, Artificial Sequence
  MF1337-CH1-IgG1 G4S linker -MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDGGGGSGGGGSEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 74 - DNA, 1117 bp, Artificial Sequence
  DNA encoding MF 1377-CH1-IgG1 H linker- MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggcccatcggtcttccccctggcaccctcctccaagtccacgtctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaagagctgcgacaagacccacaccagcccccccagccccgctcccgagctgctgggcggcgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 75 - protein, 372 aa, Artificial Sequence
  MF 1377-CH1-IgG1 H linker-MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPAPELLGGEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 76 - DNA, 1087 bp, Artificial Sequence
  DNA encoding MF1377-CH1-IgG2A MH linker -MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggccccagcgtgttccccctggccccctgcagccggagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgacggtgcccagcagcaacttcggcacccagacctacacctgcaacgtggaccacaagcccagcaacaccaaggtggacaagaccgtggagcggaagagcagcgtggagagcccccccagccccgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 77 - protein, 362 aa, Artificial Sequence
  MF1377-CH1-IgG2A MH linker -MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSSVESPPSPEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 78 - DNA, 1096 bp, Artificial Sequence
  DNA encoding MF1377-CH1-IgG1 MH linker- MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggcccatcggtcttccccctggcaccctcctccaagtccacgtctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaagagctgcgacaagacccacaccagcccccccagccccgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 79 - protein, 365 aa, Artificial Sequence
  MF1377-CH1-IgG1 MH linker-MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTSPPSPEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 80 - DNA, 1081 bp, Artificial Sequence
  DNA encoding MF1377-CH1-IgG1 UH linker-MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggcccatcggtcttccccctggcaccctcctccaagtccacgtctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaagagctgcgacaagacccacaccgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 81 - protein, 360 aa, Artificial Sequence
  MF1377-CH1-IgG1 UH linker-MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 82 - DNA, 1105 bp, Artificial Sequence
  DNA encoding MF1377-CH1-IgG2 H linker-MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggccccagcgtgttccccctggccccctgcagccggagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgacggtgcccagcagcaacttcggcacccagacctacacctgcaacgtggaccacaagcccagcaacaccaaggtggacaagaccgtggagcggaagagcagcgtggagagcccccccagccccgccccccccgtggccggcgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 83 - protein, 368 aa, Artificial Sequence
  MF1377-CH1-IgG2 H linker-MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSSVESPPSPAPPVAGEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 84 - DNA, 1105 bp, Artificial Sequence
  DNA encoding MF1377-CH1-IgG2B H linker-MF1122
ggcccagccggccatggccgaggtgcagctggtggagactggggctgaggtgaagaagccgggggcctcagtgaaggtctcctgcaaggcttctgactacatcttcaccaaatatgacatcaactgggtgcgccaggcccctggacaagggcttgaatggatgggatggatgagcgctaacactggaaacacgggctatgcacagaagttccagggcagagtcaccatgaccagggacacgtccataaacacagcctacatggagctgagcagcctgacatctggtgacacggccgtttatttctgtgcgaggagtagtcttttcaagacagagacggcgccctactatcacttcgctctggacgtctggggccaagggaccacggtcaccgtctccagtgctagcaccaagggccccagcgtgttccccctggccccctctagccggagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgacggtgcccagcagcaacttcggcacccagacctacacctgcaacgtggaccacaagcccagcaacaccaaggtggacaagaccgtggagcggaagtgcagcgtggagagcccccccagccccgccccccccgtggccggcgaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtacccttgtcaccgtctcgagt

SEQ ID NO: 85 - protein, 368 aa, Artificial Sequence
  MF1377-CH1-IgG2B H linker-MF1122
AQPAMAEVQLVETGAEVKKPGASVKVSCKASDYIFTKYDINWVRQAPGQGLEWMGWMSANTGNTGYAQKFQGRVTMTRDTSINTAYMELSSLTSGDTAVYFCARSSLFKTETAPYYHFALDVWGQGTTVTVSSASTKGPSVFPLAPSSRSTSESTAALGCLVKDYFPEPVTVSWNSGALTSGVHTFPAVLQSSGLYSLSSVVTVPSSNFGTQTYTCNVDHKPSNTKVDKTVERKCSVESPPSPAPPVAGEVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVTVSS

SEQ ID NO: 86 - DNA, 348 bp, Artificial Sequence
  MF1122 DNA
gaggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggccgtgtattactgtgcaagagccctcttcacgaccatcgccatggactattggggccaaggtaccctggtcacc

SEQ ID NO: 87 - protein, 116 aa, Artificial Sequence
  MF1122 AA
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARALFTTIAMDYWGQGTLVT

SEQ ID NO: 88 - DNA, 345 bp, Artificial Sequence
  MF1025 DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtgtattactgtgcaagggccgattggtgggcgacttttgactactggggccaaggtaccctggtcacc

SEQ ID NO: 89 - protein, 115 aa, Artificial Sequence
  MF1025 AA
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARADWWATFDYWGQGTLVT

SEQ ID NO: 90 - protein, 5 aa, Artificial Sequence
  IgG middle hinge region
CPPCP

SEQ ID NO: 91 - protein, 10 aa, Artificial Sequence
  IgG helix forming sequence
EAAAKEAAAK

SEQ ID NO: 92 - protein, 12 aa, Artificial Sequence
  multivalent multimer lacking Fc region
KSCDKTHTCPPC

SEQ ID NO: 93 - protein, 12 aa, Artificial Sequence
  PT23103p15 (Linker IgG1 H)
KSCDKTHTSPPS

(19) United States
(12) Patent Application Publication
(10) Pub. No.: US 2022/0380440 A1
(43) Pub. Date: Dec. 1, 2022

(54) TRUNCATED MULTIVALENT MULTIMERS

(71) Applicant: Merus N.V., Utrecht (NL)

(72) Inventor: Cornelis Adriaan DE KRUIF, Utrecht (NL)

(73) Assignee: Merus N.V., Utrecht (NL)

(21) Appl. No.: 17/417,288

(22) PCT Filed: Dec. 30, 2019
(86) PCT No.: PCT/NL2019/050880

§ 371 Date: Jun. 22, 2021

Related U.S. Application Data
(60) Provisional application No. 62/786,806, filed on Dec. 31, 2018.

(51) Int. Cl. C07K 16/00

(52) U.S. Cl. CPC C07K 16/00; C07K 2317/31; C07K 2317/35; C07K 2317/53; C07K 2317/54; C07K 2317/55; C07K 2317/56; C07K 2317/64; C07K 2317/66

(57) ABSTRACT

The invention relates to a truncated multivalent multimer comprising two or more binding domains, wherein each binding domain binds a different antigen or epitope, and wherein two of said binding domains are paired via a hinge region, wherein the multimer lacks a CH2 or CH3 region. The present invention further comprises two polypeptides that are paired at or near their respective C-terminus comprising two or more disulfide bridges, wherein each of said polypeptide comprise a variable binding domain, comprising a variable region, wherein each variable region binds the same or different antigens or epitopes on an antigen.

### CROSS-REFERENCE TO RELATED APPLICATIONS

This application is a U.S. national stage filing of International Application No. PCT/NL2019/050880, filed Dec. 30, 2019; which claims priority to U.S. Provisional Application No. 62/788,806, filed Dec. 31, 2018. The entire contents of International Application No. PCT/NL2019/050880 and U.S. Provisional Application No. 62/788,806 are hereby incorporated herein by reference.

### REFERENCE TO A SEQUENCE LISTING SUBMITTED ELECTRONICALLY VIA EFS-WEB

The content of the electronically submitted sequence listing in ASCII text file (Name: 4096_0350000_Seqlisting_ST25.txt; Size: 71,915 bytes; and Date of Creation: Jun. 21, 2021) filed with the application Is incorporated herein by reference in its entirety.

### FIELD

The invention relates to multivalent multimers having two or more binding domains paired via a hinge at their C-termini, and to a method for making such multivalent multimers. The Invention further relates to constituent polypeptides of the multivalent multimers capable of binding two or more epitopes or antigens, wherein such polypeptides are paired via covalent bonds comprising a disulfide bridge.

### BACKGROUND

Multivalent antibodies, such as bispecific antibodies, capable of binding two antigens or two epitopes are known in the art. Such multivalent binding proteins can be generated using various technologies, including cell fusion, chemical conjugation or recombinant DNA techniques.

Antibodies typically are multimers comprised of four proteins, including two identical heavy chains and two Identical light chains, wherein the heavy chain is comprised of a variable domain (VH), and three constant regions (CH1, CH2, CH3), and wherein the light chain is comprised of a variable light chain domain (VL) and a constant region (CL). Typically, the light chain pairs with the heavy chain through the influence of noncovalent interactions and also via a disulfide bond. The two heavy chains pair at the hinge region via disulfide bonds and through amino acid interactions in the interface between the two CH3 domains. The pairing of the VH with VL forms an antigen binding domain, and typically variability is found in three superficial-loop forming regions in the VH and VL domains, which are the complementarity determining regions or CDRs.

Certain multivalent formats are known in the art, such as antibodies having two different binding domains, such as in bispecific antibodies, that can bind two different antigens, or two different epitopes within the same antigen. Such a format can allow for the use of calibrated binding that will allow the multivalent multimer to be selectively targeted to cells or targets that express two antigens or epitopes such as a tumor cell, whilst not targeting healthy cells expressing one antigen or to target such healthy cells expressing one antigen at lower levels. Similarly, having two different binding domains on a multivalent multimer, such as a bispecific antibody, can permit binding of different antigens, such that said multivalent multimer could be used to target both an Inhibitory and a stimulatory molecule on a single cell or on two Interacting cells to result in enhanced potency of the multivalent multimer. A multivalent format could also be used to redirect cells, for example immunomodulatory cells, that could be redirected to a tumor.

While certain multivalent antibodies have been described in the art, there Is a need in the art for new formats, and new linkers, and new methodologies for making such formats that permit the efficient production of multivalent antibody formats, for which binding domains to an array of antigens can be readily made and converted into a multivalent multimer efficiently, stably, and that are capable of binding a wide array of antigens and epitopes, including such a format that lacks (partially or entirely) an Fc region comprising a CH2 and/or CH3 region.

Generating multispecific formats that lack an Fc such as a F(ab′)2 or F(ab′)n (where n=two or more) format may provide benefits over existing multispecific antibodies. For example, where engagement or redirecting of immunomodulatory cells may be desired via targeting such a cell and an antigen of Interest, the presence of an Fc component on such a targeting moiety may, for certain applications, adversely impact efficacy.

Similarly, a F(ab′)n multispecific format may be desirable compared to a full length format where a smaller size is preferred, including for potential to infiltrate solid tumors and/or to provide a shorter half-life where such a feature may benefit dosing regimens. Importantly, engineering a F(ab′)n that contains more than two binding domains having different targets has traditionally been time-consuming, inefficient, and/or costly. For example, it has been known in the art to generate F(ab′)2 moieties through a variety of ways, each of which has drawbacks where a goal is to generate large production of homogeneous batches of such a moiety for therapeutic application.

One way to generate F(ab′)2 known in the art has been by chemical means. Nisonoff and Rivers employed the production of chemical pairing several decades ago. Nisonoff A, Rivers M M (1961) Arch Biochem Biophys 93:480. Subsequently, a variety of methods have been developed that use homobifunctional and heterobifunctional chemical reagents. To date, no chemical methodology exists that can efficiently or feasibly be used as an approach for the development of a therapeutic multispecific F(ab′)n candidate, due to the time, cost, low quantity and heterogeneity that may be produced through such chemical means. Similarly, such means of chemical synthesis of F(ab′)n moieties have also been disfavored due to the use of synthetic means of pairing the two Fab domains, which raise separate concerns pertaining to uses for therapeutic applications, as well as production issues.

For example, a F(ab′)2 can be created using o-PDM. The Fab′ fragment of antibody “A” Is reacted with o-PDM, resulting in the vicinal dithiols complexed with o-PDM (R), and one of the SH groups bound to o-PDM with a free maleimide group remaining. The Fab A-o-PDM Is reacted with a free Fab′ “B” fragment, resulting in a thioether bond between two Fabs. It should be noted that difficulty associated with these synthetic means of generation include difficulty in purifying such entities to homogeneity, and constructing such moieties without altering the hinge region of the human antibody, which can lead to immunogenicity and lack of stability in vivo.

Indeed, while such artificial antibodies were generated during the 2000s and clinical trials with chemically paired Fabs were conducted for the treatment of various types of cancer, the concept appears to have been dropped including due to their lack of feasibility.

Another means of generating F(ab′)2 moeities has been through partial proteolytic digestion of IgGs with non-specific proteases such as papain. Such enzymes may cut the hinge region of IgG antibodies containing the disulfide bonds pairing the heavy chains, but also below the site of the disulfide bond between the light and the heavy chain. Such techniques have suffered from the presence of undigested IgG, over-digestion, and lack of reproducibility. The use of such nonspecific proteases has also been employed as a means of generating F(ab)2 moeities via digesting full length antibodies to cleave the Fc. These techniques also have been time-consuming and required IgG-specific optimizations and the inhibition of the protease or the purification of the Fabs to prevent their degradation. Moreover, these digestions have led to heterogenous populations of moieties overly digested, insufficiently digested, and without precision as to the cleavage site, rendering the use of such moieties for research and therapeutic application impractical. It has been difficult to control papain digestion such that only the upper hinge region Is cleaved, and not also at alternative sites, which can occur depending on the structure and flexibility of the antibody being cleaved. Accordingly, digesting multivalent antibodies having two or more binding domains via papain is inadequate and infeasible for certain applications. Similarity, pepsin has been employed for cleaving antibodies below the hinge region, with the goal of obtaining an intact F(ab′)2. The drawbacks with pepsin have been similar to papain.

Recently, enzymes have been described in the literature capable of cleaving the Fab portions away from the Fc of a full length immunoglobulin to generate individual Fab moieties or F(ab′)2. Such enzymes have been described for limited characterization applications, including for evaluating differences in avidity versus affinity or analyzing molecular weight or composition of constituent parts of a given multimer.

Accordingly, there is a need for new and useful formats for multivalent antigen-binding multimers having two or more human variable regions and linkers for the production of truncated multivalent multimers, lacking all or a portion of an Fc, having a natural (non-chemically synthesized) hinge pairing or bridging the Fab regions (F(ab′)n), and which can be efficiently and homogenously generated. Described herein, is the generation of truncated multivalent multimers, lacking an Fc region, capable of targeting antigens, and capable of use for therapeutic applications.

### SUMMARY

The present invention is directed to a multivalent multimer comprising two or more binding domains, wherein each binding domain binds a different antigen or epitope or the same antigen or epitope, and wherein two of said binding domains are paired via a hinge region, wherein the multimer lacks a constant domain, including a CH2 or CH3 region.

The present invention further comprises two polypeptides that are paired at or near their respective C-terminus comprising a disulfide bridge, wherein each of said polypeptides comprises a variable region, wherein each variable region binds the same or different antigens or epitopes on an antigen. Said binding domains preferably are themselves paired with a variable region to form a binding domain, typically VH-CH1 paired with VL-CL, wherein the VL or VH is a common chain shared in each binding domain of the multimer:

In one embodiment, the multivalent multimer comprises three or more human heavy chain variable regions comprising a short arm of a variable region (VH1) and a long arm comprising two variable regions (VH2 and VH3). In another embodiment, each heavy chain comprises an scFv. In another embodiment, each heavy chain region comprises a CH1 domain. In another embodiment, each heavy chain variable region is paired with a common light chain (VLc). In another embodiment, the common light chain comprises VL-CL. In another embodiment, the common light chain comprises the sequence of SEQ ID NO:1. In another embodiment, each heavy chain of the multimer comprises a common variable region (VHc).

In one embodiment, two of the variable regions, preferably the heavy chain variable regions, are linked via the variable region and the CH1 domain (see the hashed line in FIG. 1). In another embodiment, the variable regions are linked via a polypeptide linker. In another embodiment, a linker connecting the variable regions on a polypeptide comprises a sequence selected from the group comprising SEQ ID NOs: 2-25 or a polypeptide having at least about 85% identity to one of said sequences. In another embodiment, the linker is short, long, charged, rigid or flexible. In another embodiment, the amino acid sequence of the linker comprises a naturally-occurring sequence or comprises a sequence derived from a naturally-occurring sequence. In another embodiment, the linker comprises a middle hinge region sequence. In another embodiment, the linker comprises an upper and a lower hinge sequence. In another embodiment, the linker comprises a helix-forming sequence.

In another embodiment, two of the two or more said heavy chain variable regions are paired to each other (dimerize) at or near their respective C-terminus, preferably comprising two or more disulfide bridges, wherein said disulfide bridges are the same or substantially the same as those of a natural IgG antibody present between the CH1 and CH2 region. It is understood in the art that the number of hinge disulfide bonds varies among the immunoglobulin subclasses, each of which are encompassed by the invention described here (Papadea and Check 1989).

In one embodiment, each variable region of the multimer specifically binds a different epitope.

In another embodiment, the multivalent multimer binds at least two different antigens.

The invention is also directed to a method of producing a multivalent multimer comprising:

obtaining a panel of antibodies comprising a common light variable region and rearranged heavy chains that specifically bind to two or more targets;

integrating into a host cell a nucleic acid encoding the common light chain variable region and two or more rearranged heavy chains, wherein two of said rearranged heavy chains comprise a constant region comprising CH1, CH2 and/or CH3 domain capable of pairing;

cultivating the host cell under conditions to provide for expression of an intact multivalent multimer comprising the common light chain and two or more rearranged heavy chains, wherein two of said rearranged heavy chains are paired between the CH1 and CH2 domain of each of said two rearranged heavy chains; and

treating the intact multivalent multimer with an enzyme that cleaves the CH2 and/or CH3 region from each of the two said rearranged heavy chains, maintaining the pairing of the two said rearranged heavy chains to form the multivalent multimer.

In another embodiment disclosed herein, a panel of antibodies Is obtained by immunizing a transgenic animal comprising a nucleic acid encoding a common light chain variable region and an unrearranged heavy chain variable region with a target.

In another embodiment disclosed herein, the pairing is via two or more disulphide bridges.

In one embodiment, the heavy chain constant region of the two of said rearranged heavy chains comprising a CH1, CH2 and/or CH3 domain comprises a modification to promote heterodimerization. In another embodiment, the modification is in the immunoglobulin CH2 or CH3 regions. In another embodiment, the modification is a knob into hole modification, electrostatic modification, or DEKK modification to the respective two heavy chains. In another embodiment, the multimer expressed by the host cell comprises a first CH3 domain that dimerizes with a second CH3 domain, the first of which comprises an amino acid residue lysine at positions 351 and 368 or at positions corresponding thereto and the second of which comprises the amino acid residues of aspartic acid at 351 and glutamic acid at 368 or at positions corresponding thereto, according to EU numbering.

In another embodiment, the host cell is integrated with a nucleic acid encoding two or more different light chain variable regions capable of binding different antigens, and the two or more heavy chains variable regions are common, such that the multispecific multimer's ability to bind two or more antigens or epitopes is contributed by the different binding specificity of the light chain variable regions.

In one embodiment, the common variable regions are encoded by a nucleic acid that Is obtained from, derived from or based on a nucleic acid encoded by a transgenic animal, preferably a rodent, comprising a nucleic acid in its germline that encodes a rearranged variable chain.

In one embodiment, the method further comprises recovering the multivalent multimer.

In one embodiment, the enzyme that cleaves the CH2 and/or CH3 domains of said two heavy chains is tagged, such that it can be removed via affinity chromatography, and the mixture of enzyme, multivalent multimer and constant domain fragments Is then purified. In a further embodiment, the enzyme is charged such that it may be removed from the mixture of multivalent multimers and cleaved constant domains via charge-based chromatography.

The present invention is also directed to a multivalent multimer produced or obtainable by the methods of the invention.

The present invention is also directed to a cell which comprises a nucleic acid encoding polypeptides which are capable of assembly into a multivalent multimer of the invention.

The present invention Is also directed to a pharmaceutical composition which comprises a multivalent multimer of the invention and a pharmaceutically acceptable carrier and/or diluent.

The present invention is also directed to a method of treating a subject suffering from a medical indication comprising administering to the subject a therapeutically effective amount of a multivalent multimer of the invention.

The present invention is also directed to a multivalent multimer of the invention for use in therapy.

### BRIEF DESCRIPTION OF THE DRAWINGS

FIG. 1a: Sets out an exemplary format of multivalent multimers previously described, and FIG. 1b, sets out an exemplary format of an invention disclosed herein. The multivalent multimers of an invention disclosed herein comprise two polypeptides paired at their respective C-terminus via two or more disulfide bridges.

FIG. 2a-e: SDS-PAGE analysis of IgG, Fab, F(ab′)2 under reducing and non-reducing conditions.

FIG. 3: Heregulin-dependent MCF-7 proliferation assay demonstrating efficacy of the bispecific F(ab′)2 generated via methods disclosed herein.

FIG. 4a: Sets out an exemplary multispecific format previously disclosed, comprising a common light chain, three distinct rearranged heavy chains capable of binding different antigens, wherein two of said distinct heavy chains are paired via DEKK heterodimerization. Reference is made to WO 2019/190327, which is incorporated by reference herein. FIG. 4b: Sets out an exemplary multivalent multimer of an invention disclosed herein, comprising at 4b1, a F(ab′)3, wherein two heavy chains are paired at their respective C-terminus via two disulfide bridges. Also depicted at 4b2 is a 2Fab′ (wherein the disulphide bridge pairing the heavy chains is cleaved, and two Fabs are connected via a linker described herein), and at 4b3, a Fab. As described herein, 2Fab′ and Fab are produced when a trivalent multimer Is cleaved at the region between CH1 and CH2 for each respective heavy chain. This figure is not limiting, as additional binding domains could be added in a modular format to the base F(ab′)2 moiety by adding linkers to the N-terminal regions of either the VL or VH regions, connecting a CH1-VH or CL-VL domain, which is capable of pairing with a cognate domain to form an additional binding domain.

FIG. 5a-d: SDS-PAGE analysis of trivalent IgG molecules under reducing and non-reducing conditions, and demonstrating the successful production of IgG, Fab, F(ab′)3 multimers.

### DETAILED DESCRIPTION

The invention is based on new and modular formats for multivalent multimers comprising two or more variable regions, wherein two of the variable regions comprise at their respective C-terminus ends two or more disulfide bridges that pair said variable regions as in a natural IgG antibody, wherein said multivalent multimer is capable of binding two or more different antigens or epitopes. Said multivalent multimers lack an antibody Fc region (comprising a CH2 and/or CH3 domain), and can be produced such that said multivalent multimers include F(ab′)n, where n=two or more.

These multivalent multimers differ from typical antibody fragments, such as conventional F(ab′)2, because the multivalent multimers, including F(ab)2 or F(ab′)n described herein, may bind the same or different antigens, and are not obtained by pepsin digestion of IgG followed by reduction and reoxidation of the resulting Fab′ fragments, but rather are obtained after heterodimerization pairing, including by means of DEKK engineering, followed by enzymatic cleavage of the Fc, which leaves intact the natural hinge connecting the F(ab′)n polypeptides via disulfide bridges. These multivalent multimers optionally comprise a common chain (either heavy or light) at each binding domain, and use of linkers to connect two or more of said binding domains.

These multivalent multimers have the potential advantage of a shorter half-life, which can be associated with less accumulation in the body, thereby reducing the risks that may arise from their degradation products, and quicker adjustments to antibody concentration, which may be a benefit where, for example, a therapeutic multivalent multimer has clinical efficacy, but requires rapid clearance from the body. Also, the multivalent multimers can be less immunogenic than intact antibodies or antibody binding fragments that contain synthetic components for pairing binding domains such as (scFv)2, di-scFv and diabody moieties. An invention disclosed herein of F(ab)n moieties is new and readily producible harboring a common chain at each binding domain, such as a Fab, and includes a CH1/CL pairing that increases stability, while connecting two or more Fabs via linkers, wherein said linkers, preferably do not comprise motifs recognized by a proteolytic enzyme used in the methods described herein.

### Definitions

An “antibody” is a proteinaceous molecule belonging to the immunoglobulin class of proteins, containing one or more domains that bind an epitope on an antigen, where such domains are derived from or share sequence homology with the variable region of an antibody. Antibody binding has different qualities including specificity and affinity. The specificity determines which antigen or epitope thereof is specifically bound by the binding domain. The affinity Is a measure for the strength of binding to a particular antigen or epitope. It is convenient to note here that the ‘specificity’ of an antibody refers to its selectivity for a particular antigen, whereas ‘affinity’ refers to the strength of the interaction between the antibody's antigen binding site and the epitope it binds.

Thus, the “binding specificity” as used herein refers to the ability of an individual antibody binding site to react with an antigenic determinant. Typically, the binding site of the multimer of the invention is located in the Fab domains and is constructed from a hypervariable region of a heavy and/or light chains.

“Affinity” is the strength of the interaction between a single antigen-binding site and its antigen. A single antigen-binding site of a multimer of the invention for an antigen can be expressed in terms of the disassociation constant (KD). Typically, antibodies for therapeutic applications can have affinities of up to 1×10¹⁰ M or even higher.

An “antigen” is a molecule capable of inducing an immune response (to produce an antibody) in a host organism and/or being targeted by an antibody. At the molecular level, an antigen is characterized by its ability to be bound by the antigen-binding site of an antibody. Also mixtures of antigens can be regarded as an ‘antigen’, i.e. the skilled person would appreciate that sometimes a lysate of tumor cells, or viral particles can be indicated as ‘antigen’ whereas such tumor cell lysate or viral particle preparation comprises many antigenic determinants (e.g., epitopes). An antigen comprises at least one, but often more, epitopes.

An “epitope” or “antigenic determinant” is a site on an antigen to which an immunoglobulin or antibody specifically binds. Epitopes can be formed from contiguous amino acids or noncontiguous amino acids juxtaposed by tertiary folding of a protein (so-called linear and conformational epitopes, respectively). Epitopes formed from contiguous, linear amino acids are typically retained on exposure to denaturing solvents, whereas for epitopes formed by tertiary folding, their conformation is typically lost on treatment with denaturing solvents. An epitope can typically include 3, 4, 5, 6, 7, 8, 9, 10, 11, 12, 13, 14 or 15 amino acids in a unique spatial conformation.

The term “heavy chain” or “immunoglobulin heavy chain” includes an immunoglobulin heavy chain constant region sequence (or functional fragment thereof), and unless otherwise specified includes a heavy chain variable domain (or functional fragment thereof) from any organism. The term heavy chain variable domains include three heavy chain CDRs and four framework (FR) regions, unless otherwise specified. Fragments of heavy chains include CDRs, and FRs, and combinations thereof. A typical heavy chain Includes (from N-terminal to C-terminal), the variable domain, a CH1 domain, a hinge, a CH2 domain, and a CH3 domain. A functional fragment of a heavy chain includes a fragment that is capable of specifically recognizing an antigen and that comprises at least one CDR. Heavy chains that can be used with this invention include those, e.g., that do not selectively bind an epitope selectively bound by the cognate light chains.

The term “light chain” or “immunoglobulin light chain” includes an immunoglobulin light chain variable domain, or V_{L} (or functional fragment thereof); and an immunoglobulin constant domain, or C_{L} (or functional fragment thereof) sequence from any organism. Unless otherwise specified, the term light chain can include a light chain selected from a human kappa, lambda, and a combination thereof. Light chain variable (V_{L}) domains typically include three light chain CDRs and four framework (FR) regions, unless otherwise specified. Generally, a full-length light chain includes, from N-terminus to C-terminus, a V_{L} domain that includes FR1-CDR1-FR2-CDR2-FR3-CDR3-FR4, and a light chain constant domain. Light chains that can be used with this invention include those that do not selectively bind an epitope selectively bound by the cognate heavy chains.

Suitable light chains for use in a multivalent multimer invention include a common light chain, such as those that can be identified by screening for the most commonly employed light chains in existing antibody libraries (wet libraries or in silico), where the light chains do not substantially interfere with the affinity and/or selectivity of the epitope-binding domains of the heavy chains, but are also suitable to pair with an array of heavy chains. For example, a suitable light chain includes one from a transgenic animal, such as a transgenic rodent, comprising the common fight chain integrated into its genome and which can be used to generate large panels of common light chain antibodies having diversity at the heavy chain upon exposure to an antigen. Suitable heavy chains for use in a multivalent multimer invention may similarly include a common heavy chain.

The term “common light chain” according to the invention refers to light chains which can be identical or have some amino acid sequence differences while the binding specificity of a multimer or the invention is not affected, i.e. the differences do not materially influence the formation of functional binding regions.

It is for instance possible within the scope of the definition of common chains as used herein, to prepare or find variable chains that are not identical but still functionally equivalent, e.g., by introducing and testing conservative amino acid changes, changes of amino acids in regions that do not or only partly contribute to binding specificity when paired with a cognate chain, and the like. Such variants are thus also capable of binding different cognate chains and forming functional antigen binding domains. The term ‘common light chain’ as used herein thus refers to light chains which can be identical or have some amino acid sequence differences while retaining the binding specificity of the resulting antibody after pairing with a heavy chain. A combination of a certain common light chain and such functionally equivalent variants Is encompassed within the term “common light chain”.

The term “natural hinge region” refers to the unmodified flexible interdomain region in the central part of the heavy chains of the immunoglobulin classes, which links these 2 chains by disulfide bonds.

A hinge region is a flexible amino acid stretch in the central part of the heavy chains of the immunoglobulin classes (i.e., that portion which connects the Fab to the Fc), which pairs these two heavy chains by disulfide bonds. It Is rich in cysteine and proline amino acids, and bears little resemblance to any other Immunoglobulin region.

A “Fab domain” means a binding domain comprising a variable region, typically a binding domain comprising a paired heavy chain variable region and light chain variable region. A Fab domain can comprise constant region domains, including a CH1 and a VH domain paired with a constant light domain (CL) and VL domain. Such pairing can take place, for example, as covalent linkage via a disulfide bridge at the CH1 and CL domains.

A “modified Fab domain” means a binding domain comprising a CH1 and a VH domain, wherein the VH is paired with a VL domain and no CL domain is present. Alternatively, a modified Fab domain is a binding domain comprising a CL and a VL domain, wherein the VL is paired with a VH domain and no CH1 domain is present. In order that the CH1 or CL region can be present in a non-paired form, it can be necessary to remove or reduce the lengths of regions of hydrophobicity. CH1 regions from species of animal that naturally express single-chain antibodies, for example from a camelid animal, such as a llama or a camel, or from a shark can be used. Other examples of a modified Fab domain include a constant region, CH1 or CL, which is not paired with its cognate region and/or a variable region VH or VL, is present, which Is not paired with its cognate region.

As used herein, an “intact” antibody is one that comprises an antigen-binding site as well as a CL and at least heavy chain constant domains, CH1. CH2, and CH3. The constant domains can be native sequence constant domains (e.g., human native sequence constant domains) or amino acid sequence variant thereof.

The terms “recombinant host cell” or “host cell” refer to a cell into which exogenous DNA has been introduced. Such terms refer not only to the particular subject cell, but to the progeny of such a cell. Because certain modifications can occur in succeeding generations due to either mutation or environmental Influences, such progeny can not, in fact, be identical to the parent cell, but are still included within the scope of the term “host cell” as used herein. In an embodiment, host cells include prokaryotic and eukaryotic cells. In an embodiment, eukaryotic cells include protist, fungal, plant and animal cells. In another embodiment, host cells include, but are not limited to, the prokaryotic cell line E. coli; mammalian cell lines CHO, HEK 293, COS, NS0, SP2 and PER.C6; the Insect cell line Sf9; and the fungal cell Saccharomyces cerevisiae.

The term “Immune effector cell” or “effector ceil” as used herein refers to a cell within the natural repertoire of cells in the mammalian Immune system which can be activated to affect the viability of a target cell. Immune effector cells include cells of the lymphoid lineage such as natural killer (NK) cells, T cells including cytotoxic T cells, or B cells, but also cells of the myeloid lineage can be regarded as immune effector cells, such as monocytes or macrophages, dendritic cells and neutrophilic granulocytes. Preferable effector cells include an NK cell, a T cell, a B cell, a monocyte, a macrophage, a dendritic cell or a neutrophilic granulocyte.

“Percent (%) identity” as referring to nucleic acid or amino acid sequences herein is defined as the percentage or residues in a candidate sequence that are identical with the residues in a selected sequence, after aligning the sequences for optimal comparison purposes. The percent sequence identity comparing nucleic acid sequences Is determined using the AlignX application of the Vector NTI Program Advance 10.5.2 software using the default settings, which employ a modified ClustalW algorithm (Thompson, J. D., Higgins, D. G., and Gibson T. J. (1994) Nuc. Acid Res. 22: 4673-4880), the swgapdnarnt score matrix, a gap opening penalty of 15 and a gap extension penalty of 6.68. Amino acid sequences are aligned with the AlignX application of the Vector NTI Program Advance 11.5.2 software using default settings, which employ a modified ClustalW algorithm (Thompson, J. D., Higgins, D. G., and Gibson T. J., 1994), the blosum62mt2 score matrix, a gap opening penalty of 10 and a gap extension penalty of 0.1.

Herein, the term “connected” or “linked” refers to domains which are joined to each other by way of peptide bonds at the primary amino acid sequence. For example, a heavy chain of a variable region portion comprising VH-CH1-CH2-CH3 can be connected to a heavy chain of an additional binding domain VH-CH1 (or an additional binding domain to an additional binding domain) via a linker (connecting the heavy chain of the additional binding domain at the CH1 to the VH region of the variable region portion), which together constitutes one polypeptide chain. Similarly, a CH1 domain can be connected to a variable heavy region and a CL domain can be connected to a variable light region. The term “linker” means an amino acid residue or a polypeptide comprising two or more amino acid residues joined by peptide bonds that are used to fink two polypeptides

“Pairing” refers to interactions between the polypeptides constituting a multivalent multimer of the invention such that they can multimerize. For example, an additional binding domain can comprise a heavy chain region (VH-CH1) paired to a light chain region (VL-CL), where the CH1 and CL pair to form said binding domain. Similarly, two heavy chain polypeptides, each comprising a variable region, CH1, CH2, and/or CH3 domain may be paired together between each polypeptide's respective CH1 and CH2 domain via the formation of two or more disulfide bonds as occurs for IgG1 (or more disulfide bonds as in, for example, IgG3). Two heavy chain polypeptides may further be paired at the CH3 domains. As described herein, pairing of antibody domains (e.g., heavy and light) may further occur due to noncovalent interactions and also via disulfide bonds, and can be engineered through techniques disclosed herein and by methods known in the art.

Throughout the present specification and the accompanying claims, the words “comprise”, “include” and “having” and variations such as “comprises”, “comprising”. “includes” and “including” are to be interpreted inclusively. That is, these words are intended to convey the possible inclusion of other elements or integers not specifically recited, where the context allows.

The articles “a” and “an” are used herein to refer to one or to more than one (i.e. to one or at least one) of the grammatical object of the article. By way of example, “an element” can mean one element or more than one element.

Different Formats of the Multivalent Multimer

The invention provides a truncated multivalent multimer which Is capable of binding to its target or targets via its two or more binding domains. A multivalent multimer of the invention can comprise two or more variable regions, or a portion thereof, capable of binding an antigen. Importantly, the multimer lacks all or a portion of an Fc region, preferably the entire Fc. The multimer of an invention disclosed herein comprises two heavy chain regions paired at their respective C-terminus via a hinge, preferably a natural hinge, and more preferably comprising two or more disulfide bonds.

In some embodiments, the multimer comprises one or more additional binding domains. In one embodiment, the multimer comprises a Fab domain comprising a VH-CH1 region paired to a VL-CL region.

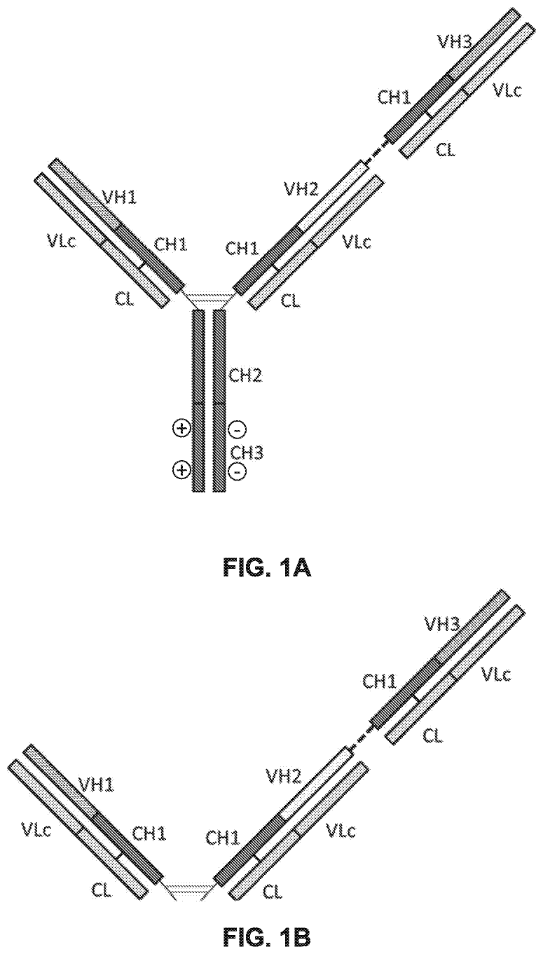

Said multivalent multimer may thus comprise three VH regions, and three VL regions. Either of the VH or VL can be a common variable region (VHc or VLc) paired to a rearranged variable region of the cognate chain, or one in which binding specificity to an epitope or antigen is conferred by the non-common chain. For example, the three VL regions can be a common chain (VLc), and each VH region (VH1-VH3) can comprise a rearranged variable region, wherein said VH1, VH2 and VH3 regions can bind the same epitope or three different epitopes. As shown in FIG. 1a. VH1 is used to refer to the short arm, VH2 and VH3 are used to refer to the long arm, where VH2 is the interior arm which is paired to VH1 at their respective C-terminus, and VH3 Is used to refer to the distal arm.

Wherein, the multivalent multimer comprises a common light chain (VLc) and three heavy chain variable regions (VH1-VH3), the additional Fab domain comprised of a VH3-CH1 paired with a VLc-CL can be connected to the variable region via a linker positioned between a VH2 region or VLc and CH1 of the additional Fab domain or the CL of the additional Fab domain.

In another embodiment, the individual polypeptides that make up the multivalent multimer can mix heavy and light chains within the same protein. A multivalent multimer of the invention can comprise a modified Fab domain. The modified Fab domain can comprise a modified CH1 such that it does not need to pair with a CL. For example, the CH1 could be a camelid CH1 or based on a camelid CH1, or be modified to lack hydrophobic residues through techniques known in the art. Each VH or VL can be a common or rearranged variable region.

A multivalent multimer of the invention can comprise a modified Fab domain that does not need to pair with a CH1. For example, the CL could be engineered to remove hydrophobic regions. Each VH or VL of the modified Fab domain can be a common or rearranged variable region. The additional modified Fab domain can be connected to the variable region portion via a linker positioned between the VL of the variable region portion and CL of the modified Fab domain. The VH and VL of the modified Fab domain can be paired via a cysteine bridge. A multivalent multimer of the invention can comprise a modified Fab domain comprising a modified CL that does not need to pair with a CH1.

### Generation or the Multivalent Multimers

In one embodiment, the multivalent multimers can be produced by enzymatic digestion of intact multivalent antibodies, or cleavage of said multivalent antibodies at specific regions that leave a natural hinge or pairing of polypeptides of said multivalent multimer intact. The intact antibodies can be a full length immunoglobin, for example a full length IgG, IgA, IgE, IgD or IgM portion, but preferably IgG, and more preferably IgG1.

The heavy chains of the multivalent multimers can be designed to preferentially pair through techniques known to those of skill in the art, such as engineering DEKK modifications in the CH3 regions of the intact antibody. See WO2013/157954 and De Nardis et al., J. Biol. Chem. (2017) 292(35) 14706-14717, incorporated herein by reference, demonstrating engineering in the CH3 region for driving heterodimerization of the heavy chains. Alternative approaches for driving heterodimerization which can be used in the invention include the knob-in-hole format (WO1998/050431) and use of charge engineering (Gunasekaran, JBC 2010, vol 285, pp 19637-19646), and other suitable techniques known in the art.

### Linkers for Use in the Multivalent Multimer Format

A multivalent multimer of the invention can comprise one or more linkers which connect the one or more variable regions. The linker together with the binding domain to which the linker is connected may determine, at least in part, the functionality of the multivalent multimer.

The linker can comprise a hinge sequence or comprise a sequence based on a hinge sequence. Thus, the amino acid sequence of a suitable linker can comprise a naturally-occurring sequence or comprise a sequence derived from or based on a naturally-occurring sequence. The use of such sequences can help developability of multivalent antibodies of the invention and/or help to ensure low immunogenicity. For the purposes of the current application, it is preferred that the linker does not contain an enzymatic recognition site for any enzyme used to cleave the Fab or F(ab′)n from the Fc, such that the enzyme would similarly cleave the linkage between binding domains. For example, where a truncated multivalent multimer is produced comprising three Fab domains, comprising a common light chain (VLc) and three heavy chain variable regions (VH1-VH3), it is preferred that the linker connecting the VH3-CH1 paired with a VLc-CL to a VH1 or VH2 region or VLc region, does not include an amino acid motif recognized by an enzyme capable of cleaving an Fc from a Fab, 2Fab′ or F(ab′)3.

Accordingly, a suitable linker to connect the one or more additional binding domains to the two or more variable regions can be derived from an IgG or IgA hinge sequence. The linker region can be based on an IgG1 hinge region, an IgG2 hinge region, an IgG3 hinge region or an IgG4 hinge region.

Typically, the type of the hinge region used is matched with the type of the constant region, for example the CH1, of the additional Fab domain to which the linker is connected. That is to say, if a linker Is based on a sequence or sequences from a IgG1 hinge region, the CH1 of the additional Fab domain to which it is connected Is a CH1 from a IgG1.

A linker of an antibody can be based on an upper, middle or lower hinge region, or a subset of such a region.

The IgG1 hinge region has the sequence: EPKSCDKTHTCPPCPAPELLGG (SEQ ID NO: 26).

The upper hinge region is defined as: EPKSCDKTHT (SEQ ID NO: 3)

The middle hinge region is defined as: CPPCP (SEQ ID NO: 27)

The lower hinge region is defined as: APELLGG (SEQ ID NO: 28)

Thus, in a multimer of the invention, the linker can comprise one or more of these sequences and/or a sequence derived from or based on one or more or these sequences.

The IgG2 hinge region has the sequence: ERKCCVECPPCPAPPVAG (SEQ ID NO: 29).

The upper hinge region is defined as: ERKCCVE (SEQ ID NO: 30)

The middle hinge region is defined as: CPPCP (SEQ ID NO: 27)

The lower hinge region is defined as: APPVAG (SEQ ID NO: 31)

Thus, in a multimer of the invention, the linker can comprise one or more of these sequences and/or a sequence derived from or based on one or more of these sequences.

The IgG3 hinge region has the sequence:

(SEQ ID NO: 32)
ELKTPLGDTTHTCPRCPEPKSCDTPPPCPRCPEPKSCDTPPPCPRCPEPKS
CDTPPPCPRCPAPEFLGG

The upper hinge region is defined as: ELKTPLGDTTHT (SEQ ID NO: 7)

The middle hinge region is defined as:

(SEQ ID NO: 33)
CPRCPEPKSCDTPPPCPRCPEPKSCDTPPPCPRCPEPKSCDTPPPCPRCP

The lower hinge region is defined as: APEFLGG (SEQ ID NO: 34)

The IgG4 hinge region has the sequence: ESKYGPPCPSCPAPEFLGG (SEQ ID NO: 35).

The upper hinge region is defined as: ESKYGPP (SEQ ID NO: 2)

The middle hinge region is defined as: CPSCP (SEQ ID NO: 36)

The lower hinge region is defined as: APEFLGG (SEQ ID NO: 34).

The middle region with consensus sequence CXXC connects both IgG heavy chains in the context of a wildtype IgG and is rigid. These disulfide bridges are not required for the current application and, therefore, where a linker comprises a middle hinge sequence, preferably, one or both Cys residues in the CXXC consensus are substituted, for example with a Ser residue. Thus, in one embodiment CxxC can be SxxS.

A linker suitable for use in a multivalent multimer of the invention can be one derived from or based on a middle hinge sequence, for example a sequence which comprises a middle hinge sequence, but which does not comprise a lower and/or an upper hinge sequence. A linker suitable for use in a multivalent multimer of the invention can be one derived from or based on an upper hinge sequence, for example a sequence which comprises an upper hinge sequence, but which does not comprise a lower and/or a middle hinge sequence. A linker suitable for use in a multivalent multimer of the invention can be one which does not comprise a middle hinge sequence, for example a sequence which comprises a combination of lower and upper hinge sequences.

Thus, in a multimer of the Invention, the linker can comprise one or more of these sequences and/or a sequence derived from or based on one or more of these sequences. A linker can consist essentially of a middle hinge region sequence or be derived from or based on such as sequence or consist essentially of an upper and a lower hinge region sequence or be derived from or based on such sequences.

A linker suitable for use in a multimer of the invention can be defined with reference to a sequence comprising the amino acid sequence of any linker sequence as set out herein in which from 0 to 5 amino acid insertions, deletions, substitutions or additions (or a combination thereof) is made. In some embodiments, the linker comprises an amino acid sequence comprising from 0 to 4, preferably from 0 to 3, preferably from 0 to 2, preferably from 0 to 1 and preferably 0 amino acid insertions, deletions, substitutions or additions (or a combination thereof) with respect to a linker sequence as set out herein.

A suitable linker can be from about 7 to about 29 amino acids in length, for example from about 10 to about 20 amino acids in length. However, a suitable linker can be a short linker, for example from about 7 to about 10 amino acids in length or can be a long linker, for example from about 20 to about 29 amino acids in length.

The linker can comprise an Ig hinge region or comprise a sequence derived from or based on an IgG hinge region connected to a CH1 region of the same subclass as the linker and can comprise cysteines for covalent linkage of the common light chain.

A linker suitable for use in a multimer of the invention can be derived from or based on an IgG1 hinge region, an IgG2 hinge region, an IgG3 hinge region or an IgG4 hinge region.

If a (G₄S)ₙ sequence is to be used, preferably it is used in combination with a hinge sequence from an isotype other than IgG or a subclass other than IgG1 and includes a CH1 region.

In a multimer of the invention, the linker can be rigid or flexible can comprise a charged sequence, can be straight or bent.

A rigid sequence for the purposes of this invention Is sequence having a Karplus and Schulz flexibility Prediction of about 1.015 or less. A partially flexible sequence is one having a Karplus and Schulz flexibility Prediction of from about 1.015 to about 1.04. A flexible sequence for the purposes of this invention is sequence having a Karplus and Schulz flexibility Prediction of at least about 1.015 (Karplus P A, Schulz G E. Prediction of Chain Flexibility in Proteins—A tool for the Selection of Peptide Antigens. Naturwissenschaften 1985: 72:212-3; http://tools.immuneepitope.org/bcell). The flexibility prediction Is calculated over consecutive windows of 7 residues along the sequence (1 residue step) yielding the predicted “flexibility” index per window. The overall flexibility over the linker sequence Is given as the average over the whole sequence.

Removal or substitution of Cys residues in an IgG hinge region can make a linker based on that hinge more flexible including through replacement of the Cys residue with a serine (Ser). Alternatively, a linker can be a rigid linker in view of the presence of a helix-forming sequence. Accordingly, a middle hinge region, for example the conserved CPPCP (SEQ ID NO: 90) motif, can be replaced by a helix-forming sequence, for example (EAAAK)₂ (SEQ ID NO: 91), which will result in a short rigid helix in the linker. Therefore, in a multimer of the invention, the linker can comprise a helix-forming sequence, for example comprising the amino acid sequence (EAAAK)₂ (SEQ ID NO: 91). The use of such a sequence can help to add rigidity.

A linker of the invention can comprise an amino acid sequence as set out in any one of SEQ ID NOs: 4 to 6, 8 to 12 or 14 to 25 or an amino acid sequence having at least about 90% sequence identity to any one thereto, preferably at least about 95% sequence Identity to any one thereto, more preferably at least 97% sequence identity to any one thereto, more preferably at least about 98% sequence identity to any one thereto, more preferably at least about 99% sequence identity to any one thereto.

For example, a linker suitable for use in a multivalent multimer of the invention can be defined with reference to a sequence comprising the amino acid sequence of any one of SEQ ID NOs: 2 to 25 in which from 0 to 5 amino acid insertions, deletions, substitutions or additions (or a combination thereof) is made. In some embodiments, the linker comprises an amino acid sequence having from 0 to 4, preferably from 0 to 3, preferably from 0 to 2, preferably from 0 to 1 and preferably 0 amino acid insertions, deletions, substitutions or additions (or a combination thereof) with respect to a sequence set out in SEQ ID NOs: 4 to 6, 8 to 12 or 14 to 25.

A linker suitable for use in a multivalent multimer of the invention can be defined with reference to a sequence comprising the amino acid sequence of any one of SEQ ID NOs: 2 to 25 or an amino acid sequence having at least about 85% sequence identity to any one thereto, such as at least about 90% sequence identity to any one thereto, for example at least about 95% sequence identity to any one thereto, such as at least about 98% sequence identity to any one thereto, for example at least about 99% sequence identity to any one thereto.

TABLE 1
The sequences of the 24 different linkers/
constructs and naming as used

| | | Linker |
|---|---|---|
| Linker | | size |
| name | Sequence | (aa) |
| IgG4 UH | ESKYGPP | 7 |
| | (SEQ ID NO: 2) | |
| IgG1 UH | EPKSCDKTHT | 10 |
| | (SEQ ID NO: 3) | |
| IgG2A G4SS | GGGGSGGGGS | 10 |
| | (SEQ ID NO: 4) | |
| IgG2A MH | ERKSSVESPPSP | 12 |
| | (SEQ ID NO: 5) | |
| IgG2B MH | ERKCSVESPPSP | 12 |
| | (SEQ ID NO: 6) | |
| IgG3 UH | ELKTPLGDTTHT | 12 |
| | (SEQ ID NO: 7) | |
| IgG4 MH | ESKYGPPSPSSP | 12 |
| | (SEQ ID NO: 8) | |
| IgG2A UL | ERKSSVEAPPVAG | 13 |
| | (SEQ ID NO: 9) | |
| IgG2B UL | ERKCSVEAPPVAG | 13 |
| | (SEQ ID NO: 10) | |
| IgG4 UL | ESKYGPPAPEFLGG | 14 |
| | (SEQ ID NO: 11) | |
| IgG1 MH | EPKSCDKTHTSPPSP | 15 |
| | (SEQ ID NO: 12) | |
| IgG1 G4S | EPKSCDGGGGSGGGGS | 16 |
| | (SEQ ID NO: 13) | |
| IgG2 G4SL | GGGGSGGGGSAPPVAG | 16 |
| | (SEQ ID NO: 14) | |
| IgG1 UL | EPKSCDKTHTAPELLGG | 17 |
| | (SEQ ID NO: 15) | |
| IgG2A H | ERKSSVESPPSPAPPVAG | 18 |
| | (SEQ ID NO: 16) | |
| IgG2B H | ERKCSVESPPSPAPPVAG | 18 |
| | (SEQ ID NO: 17) | |
| IgG3 ULH | ELKTPLGDTTHTAPEFLGG | 19 |
| | (SEQ ID NO: 18) | |
| IgG4 H | ESKYGPPSPSSPAPEFLGG | 19 |
| | (SEQ ID NO: 19) | |
| IgG1 H | EPKSCDKTHTSPPSPAPELLGG | 22 |
| | (SEQ ID NO: 20) | |
| IgG2A R | ERKSSVEEAAAKEAAAKAPPVAG | 23 |
| | (SEQ ID NO: 21) | |
| IgG2B R | ERKCSVEEAAAKEAAAKAPPVAG | 23 |
| | (SEQ ID NO: 22) | |
| IgG4 R | ESKYGPPEAAAKEAAAKAPEFLGG | 24 |
| | (SEQ ID NO: 23) | |
| IgG1 R | EPKSCDKTHTEAAAKEAAAKAPELLGG | 27 |
| | (SEQ ID NO: 24) | |
| IgG3 R | ELKTPLGDTTHTEAAAKEAAAKAPEFLGG | 29 |
| | (SEQ ID NO: 25) | |

### Use of Linkers to Pair Regions of an Additional Binding Domain

The linkers used herein can connect the one or more variable regions to at least one additional binding domain. In addition, where the at least one additional binding domain Is a Fab domain or is comprised of pairing of a heavy chain variable region and a light chain variable region, the linker can pair the heavy and light chains via covalent linkage, typically via a disulfide bridge. Thus, where a linker connects variable regions, it forms part of the primary amino acid sequence of a polypeptide, for example, VH-1-CH1-Linker-VH2-CH1. In contrast, where a linker pairs two variable domains, it bridges these domains together, including for example, by producing contact points, a covalent bond, for example, a disulfide bond between the two variable domains, which constitute separate polypeptides.

The disulfide bridge can form between a cysteine residue in the linker and a variable region of the additional binding domain(s). Such pairing caused by the linker can apply to an additional binding domain, comprising a Fab domain comprising a common light chain and a counterpart rearranged heavy chain variable region or comprising a common heavy chain and a counterpart rearranged light chain variable region.

Table 2 illustrates how a linker sequence can be connected to CH1 and VH2 regions.

TABLE 2
The underlined sequence is the linker sequence;
the flanking sequences are the CH1 region of
the additional Fab's CH1 region and a VH2
region

| | | Linker sequences (underlined) contain- |
|---|---|---|
| | | ing CH1 region and VH sequence preced- |
| | | ing and following the linker sequence |
| | | respectively. In the control IgG1 |
| | | sequence the CH2 region is present |
| # | Name | (underlined) |
| 1 | IgG1 | NVNHKPSNTKVDKRVEPKSCDKTHTSPPSPAPELLGGEV |
| | H | QLVESGGGVVQPG (SEQ ID NO: 37) |
| 2 | IgG1 | NVNHKPSNTKVDKRVEPKSCDKTHTSPPSPEVQLVESGG |
| | MH | GVVQPG (SEQ ID NO: 38) |
| 3 | IgG1 | NVNHKPSNTKVDKRVEPKSCDKTHTEVQLVESGGGVQPG |
| | UH | (SEQ ID NO: 39) |
| 4 | IgG1 | NVNHKPSNTKVDKRVEPKSCDGGGGSGGGGSEVQLVESG |
| | G4S | GGVVQPG (SEQ ID NO: 40) |
| 5 | IgG1 | NVNHKPSNTKVDKRVEPKSCDKTHTEAAAKEAAAKAPEL |
| | R | LGGEVQLVESGGGVVQPG (SEQ ID NO: 41) |
| 6 | IgG1 | NVNHKPSNTKVDKRVEPKSCDKTHTAPELLGGEVQLVES |
| | UL | GGGVVQPG (SEQ ID NO: 42) |
| 7 | IgG2A | NVDHKPSNTKVDKTVERKSSVESPPSPAPPVAGEVQLVE |
| | H | SGGGVVQPG (SEQ ID NO: 43) |
| 8 | IgG2A | NVDHKPSNTKVDKTVERKSSVESPPSPEVQLVESGGGVV |
| | MH | QPG (SEQ ID NO: 44) |
| 9 | IgG2A | NVDHKPSNTKVDKTVERKSSVEAPPVAGEVQLVESGGGV |
| | UL | VQPG (SEQ ID NO: 45) |
| 10 | IgG2B | NVDHKPSNTKVDKTVERKCSVESPPSPAPPVAGEVQLVE |
| | H | SGGGVVQPG (SEQ ID NO: 46) |
| 11 | IgG2B | NVDHKPSNTKVDKTVERKCSVESPPSPEVQLVESGGGVV |
| | MH | QPG (SEQ ID NO: 47) |
| 12 | IgG2B | NVDHKPSNTKVDKTVERKCSVEAPPVAGEVQLVESGGGV |
| | UL | VQPG (SEQ ID NO: 48) |
| 13 | IgG2A | NVDHKPSNTKVDKTVGGGGSGGGGSAPPVAGEVQLVESG |
| | G4SL | GGVVQPG (SEQ ID NO: 49) |
| 14 | IgG2A | NVDHKPSNTKVDKTVGGGGSGGGGSEVQLVESGGGVVQP |
| | G4SS | G (SEQ ID NO: 50) |
| 15 | IgG2A | NVDHKPSNTKVDKTVERKSSVEEAAAKEAAAKAPPVAGE |
| | R | VQLVESGGGVVQPG (SEQ ID NO: 51) |
| 16 | IgG2B | NVDHKPSNTKVDKTVERKCSVEEAAAKEAAAKAPPVAGE |
| | R | VQLVESGGGVVQPG (SEQ ID NO: 52) |
| 17 | IgG3 | NVNHKPSNTKVDKRVELKTPLGDTTHTAPEFLGGEVQLV |
| | ULH | ESGGGVVQPG (SEQ ID NO: 53) |
| 18 | IgG | NVNHKPSNTKVDKRVELKTPLGDTTHTEVQLVESGGGVV |
| | UH | QPG (SEQ ID NO: 54) |
| 19 | IgG3 | NVNHKPSNTKVDKRVELKTPLGDTTHTEAAAKEAAAKA |
| | R | PEFLGGEVQLVESGGGVVQPG (SEQ ID NO: 55) |
| 20 | IgG4 | NVDHKPSNTKVDKRVESKYGPPSPSSPAPEFLGGEVQLV |
| | H | ESGGGVVQPG (SEQ ID NO: 56) |
| 21 | IgG4 | NVDHKPSNTKVDKRVESKYGPPSPSSPEVQLVESGGGVV |
| | MH | QPG (SEQ ID NO: 57) |
| 22 | IgG4 | NVDHKPSNTKVDKRVESKYGPPAPEFLGGEVQLVESGGG |
| | UL | VVQPG (SEQ ID NO: 58) |
| 23 | IgG4 | NVDHKPSNTKVDKRVESKYGPPEVQLVESGGGVVQPG |
| | UH | (SEQ ID NO: 59) |
| 24 | IgG4 | NVDHKPSNTKVDKRVESKYGPPEAAAKEAAAKAPEFLGG |
| | R | EVQLVESGGGVVQPG (SEQ ID NO: 60) |
| 25 | IgGI | NVNHKPSNTKVDKRVEPKSCDKTHTCPPCPAPELLGGPS |
| | hinge | VFLFPPKPKDTLM (SEQ ID NO: 61) |

Note, the VH2 sequence, following the linker (underscored above) may vary, depending on the specific variable region used. In other embodiments, the sequence following the linker may be a light chain variable region, including a common light chain.

### Multivalency and Multispecificity

Where the two or more variable regions bind different antigens, the first and second antigens can be two different molecules or moieties that are located on one cell or on different cell types. Antibodies comprising two binding domains that mediate cytotoxicity by recruiting and activating endogenous immune cells are an emerging class of antibody therapeutics. This can be achieved by combining antigen binding specificities for target cells (i.e., tumor cells) and effector cells (e.g., T cells, NK cells, and macrophages) in one molecule (see, for example, WO2014/051433). A multimer of the invention comprises at least two binding domains. A multivalent multimer comprising three or more binding domains can target one, two, three or more tumor associated antigens, permitting a specific targeting of deleterious cells over healthy cells. For example, one binding domain or two binding domains of the multivalent multimer can bind an antigen on an aberrant (tumor) cell, whereas a second or third binding domain of the multivalent multimer can bind an antigen on an immune effector cell that can cause directed killing of the tumor cell expressing the one or more tumor associated antigens. Alternatively, two binding domains of the multivalent multimer can bind specifically to two different epitopes on an identical antigen or different antigens expressed on tumor cells while the affinities of these arms are attenuated to mitigate binding to cells expressing only one antigen or where only one binding domain of the multivalent multimer is engaged. Alternatively, three binding domains of the multivalent multimer of the invention can bind to three different antigens or to Identical antigens, but at different epitopes of immune effector cells.

Similarly, a multivalent multimer comprising three or more binding domains can bind a functional target such as a ligand or enzyme, triggering a biological response or blocking the function of the target, resulting in inhibitory or agonistic cellular activity. At least one binding domain of a multivalent multimer of the invention is connected via a linker to a binding domain of the variable region portion.

Where the binding domain of at least one of the variable regions is a Fab domain, this can take the form, for example, of VH-CH1-linker-VH-CH1, wherein the linker connects the heavy chain of one variable region to the at least one additional binding domain, preferably a Fab domain.

Alternatively, this can take the form, for example, of VL-CL-linker-VL-CL, wherein the linker connects the light chain of one variable region to the at least one additional binding domain, preferably a Fab domain.

An additional binding domain, such as a Fab domain, can be connected to two variable regions, each via a separate linker. The two or more linkers connecting the additional variable regions or additional binding domains can be the same or different. Further, the linkers can allow pairing of the cognate chains of the binding domain.

If a multimer of the invention comprises more than one linker, those linkers can be the same or different or a combination thereof. An example of the latter situation is where a multivalent multimer comprises three linkers, two of which are the same and a third which is different (from the other two).

Further a variable region connected via a linker to another variable region, can itself be attached to a variable region connected via a linker described herein, wherein the other variable regions can be extended in a modular fashion by connecting through a linker to an additional binding domain, and connecting that variable region to a second additional variable region through a linker and so on.

In this way a multimer of the invention can be capable of binding two, three or more epitopes.

A multimer of the invention can be capable of binding two, three or more antigens.

A multimer of the invention can comprise two or more variable regions, such as two or more Fab domains, which are capable of binding to different epitopes on one antigen.

In one embodiment, a multimer of the invention comprises at least three binding domains, such as three or more Fab domains of which at least two Fabs are different.

Another aspect of the invention comprises a multivalent multimer comprising at least three Fab domains and therefore is capable of binding to three epitopes which are typically all different from each other.

A multimer of the invention can also be multispecific. Multivalent Indicates that the antibody has at least two binding domains and therefore has at least two antigen-binding sites. Multispecific Indicates that the antibody is capable of binding at least two different epitopes, for example two different antigens or two epitopes on the same antigen. Trispecific indicates that the antibody is capable of binding three different epitopes. Quadraspecific indicates that the antibody is capable of binding four different epitopes and so on.

A multimer of the invention can bind target epitopes which are located on the same target. This can allow for more efficient counteraction of the (biological) function of said target molecule as compared to a situation wherein only one epitope is targeted. For example, a multimer of the invention can simultaneously bind to 2 or 3 or more epitopes present on an antigen cell, e.g., growth factor receptors or soluble molecules critical for tumors cells to proliferate, thereby effectively blocking several independent signaling pathways leading to uncontrolled proliferation.

Any combination of at least two multimers of the invention can simultaneously bind to 2, 3, 4 or more epitopes present on a target molecule, such as a growth factor receptor or soluble molecule. In a combination of at least two multimers of the invention, two multimers may share at least one common binding domain.

The target moiety can be a soluble moiety or can be a membrane-bound moiety or can be a moiety present on a cell-surface that internalizes upon binding.

The target epitopes can be located on different moieties, for example on two (i.e. two or more target epitopes on a first moiety and one or more target epitopes on a second moiety) or three different moieties (i.e. at least one target epitope on each of three moieties). In this case, each of the different target moieties can either be a soluble moiety or a membrane-bound moiety or a moiety present on a cell-surface that internalizes upon binding. In one embodiment, the different target moieties are soluble moieties. Alternatively, at least one target moiety Is a soluble moiety whereas and at least one target moiety Is a membrane bound moiety. In yet another alternative, all target moieties are membrane bound moieties. In one embodiment, the different target moieties are expressed on the same cell, whereas in other embodiments the different target moieties are expressed on different cells.

As a non-limiting example, any multimer of the invention or any combination of a multimer of the invention and an additional antibody can be suitable for simultaneously blocking multiple membrane-bound receptors, neutralizing multiple soluble molecules such as cytokines or growth factors for tumor cells or for neutralizing different viral serotypes or viral strains.

In one embodiment, at least one target epitope can be located on a tumor cell. Alternatively, or additionally, at least a target epitope can be located on the surface of an effector cell. This is for instance suitable for recruitment of T cells or NK cells for tumor cell killing. For instance, a multimer of the invention can be capable of recruiting immune effector cells, preferably human immune effector cells, by specifically binding to a target molecule located on immune effector cells. In a further embodiment, said immune effector cell is activated upon binding of the multimer of the invention to the target molecule. Recruitment of effector mechanisms can for instance encompass the redirection of immune modulated cytotoxicity by administering an Ig-like molecule produced by a method according to the invention that is capable of binding to a cytotoxic trigger molecule such as the T cell receptor or an Fc gamma receptor, thereby activating downstream immune effector pathways or immune effector cells.

### Common Variable Region

The multivalent multimer of the Invention can use a common chain at each of the two or more binding domains (variable regions). As described, in one embodiment multivalent multimer has a first heavy chain variable region/light chain variable region (VH/VL) combination that binds one antigen and a second VH/VL combination that binds a second antigen. Each additional binding domain can also comprise an additional VH/VL combination that binds a further epitope on an antigen.

In one embodiment, the multimer comprises two heavy chains (one or both comprising one or more additional CH1 and VH domain) and a light chain which pairs with each CH1 and VH domain. In one embodiment, the two heavy chains have compatible heterodimerization domains, and the light chain is a common light chain. In another embodiment, the multimer comprises two light chains (one or both comprising one or more additional CL and VL domain) and a heavy chain variable region which pairs with each CL and VL domain, and the heavy chain variable region comprises a common heavy chain variable region.

Where the multivalent multimer comprises a common light chain, where said light chain is expressed within a host cell that includes DNA encoding two or more heavy chain variable regions, said light chain is capable of pairing with each available heavy chains (or CH1-VH1 regions), thereby forming at least three functional antigen binding domains.

A functional antigen binding domain (variable region) Is capable of specifically binding to an epitope on an antigen. In one embodiment, a common light chain is capable of pairing with all heavy chains (or CH1-VH1 regions) produced, so that mispairing of unmatched heavy and light chains is avoided or produced at a significantly lower ratio than the multivalent multimer.

In one embodiment, the multivalent multimer of the Invention has a common light chain (variable region) that can combine with an array of heavy chain variable regions to form a multimer with functional antigen binding domains (WO2004/009618, WO2009/157771).

A common light chain (variable region) for use in the multivalent multimer of the invention is preferably a human light chain. In one embodiment, the common light chain (variable region) has a germline sequence. In one embodiment, the germline sequence Is a light chain variable region that is frequently used in the human repertoire and has good thermodynamic stability, yield and solubility. A preferred germline light chain is the human IgVκ1-39*01/IGJκ1*01 and human constant region (SEQ ID NO: 1). The nucleic acid encoding the common light chain variable region is preferably the rearranged germline human kappa light chain IgVκ1-39*01/IGJκ1*01 (SEQ ID NO: 62). A common light chain preferably comprises a light chain variable region amino acid sequence of SEQ ID NOs: 63 and human light chain constant region amino acid sequence of SEQ ID NO: 64, with 0-5 amino acid Insertions, deletions, substitutions, additions or a combination thereof. The common light chain can further comprise a light chain constant region, preferably a kappa light chain constant region. A nucleic acid that encodes the common light chain (SEQ ID NO:1) can be codon optimized for the cell system used to express the common light chain protein. The encoding nucleic acid can deviate from a germ-line nucleic acid sequence.

The common light chain (variable region) for use in the multivalent antibodies of the invention can be a lambda light chain and this is therefore also provided in the context of the invention, however a kappa light chain is preferred. The common light chain of the invention can comprise a constant region of a kappa or a lambda light chain. In one embodiment, the constant region of a kappa light chain is used, preferably wherein said common light chain is a germline light chain, preferably a rearranged germline human kappa light chain comprising the IgV_{κ}I-39 gene segment, for example the rearranged germine human kappa light chain IgV_{κ}I-39*01/IGJ_{κ}I*01. Those of skill in the art will recognize that “common” also refers to functional equivalents of the light chain of which the amino acid sequence Is not identical. Many variants of said light chain exist wherein mutations (deletions, substitutions, additions) are present that do not materially influence the formation of functional binding regions.

IgVκ1-39 is short for Immunoglobulin Variable Kappa 1-39 Gene. The gene is also known as Immunoglobulin Kappa Variable 1-39: IGKV139; IGKV1-39. External Ids for the gene are HGNC: 5740; Entrez Gene: 28930; Ensembl: ENSG00000242371. A preferred amino acid sequence for IgVκ1-39 is given in SEQ ID NO: 65. This lists the sequence of the V-region. The V-region can be combined with one of five J-regions. A common light chain variable region is preferably linked to a kappa light chain constant region. In a preferred embodiment the light chain variable region used in the multivalent multimer of the invention comprises the kappa light chain IgVκ1-39*01/IGJκ1*01 or IgVκ1-39*01/IGJκ5*01. In a preferred embodiment the common light chain in the multivalent multimer is IgVκ1-39*01/IGJκ1*01.

A cell that produces a common light chain can produce for instance rearranged germine human kappa light chain IgVκ1-39*01/IGJκ1*01 and a light chain comprising the variable region of the mentioned light chain fused to a lambda constant region. Where herein reference is made to a germ-fine sequence, in one embodiment the variable region is a germ-line sequence.

A preferred common light chain for use in a multivalent multimer of the invention is one comprising the sequence set out in SEQ ID NO: 1.

The common chain for use in the multivalent antibodies of the invention can also be a heavy chain and this is therefore also provided in the context of the invention. Common heavy chains have been used in the art to make bispecific antibodies, and can be used here in making a multivalent multimer comprising three or more binding domain, two or more of said binding domains comprise a common heavy chain known in the art. For example, the use of antibody libraries in which the heavy chain variable domain is the same for all the library members and thus the diversity is based on the light chain variable domain. Such libraries are described, for example, PCT/US2010/035819, and PCT/US2010/057780, each of which is hereby incorporated by reference in its entirety. These and other techniques of generating binding domains having common heavy chains can be generated by the skilled artisan, and can be employed in the present invention to produce multivalent antibodies having novel formats disclosed herein.

### Production of a Truncated Multivalent Multimer

In one embodiment, a host cell can be co-transfected with a nucleic acid encoding two or more heavy chain variable regions and a common light chain variable region to produce a multivalent multimer, wherein two of said heavy chain variable regions comprise a constant region, including CH1, CH2 and/or CH3, which are capable of heterodimerization including via a pairing at a hinge between the CH1 and CH2 domains, and wherein said two heavy chains each comprise an amino acid sequence below said hinge recognized by a proteolytic enzyme capable of cleaving the CH2 and/or CH3 region. Alternatively, a multivalent multimer of the invention can be produced by co-transfection of Individual cells with one or more genetic constructs which together encode the two or more light chain variable regions and a common heavy chain, wherein two common heavy chains comprise a constant region, including CH1, CH2 and/or CH3, which are capable of heterodimerization including via a pairing at a hinge between the CH1 and CH2 domains, and wherein said two heavy chains each comprise an amino acid sequence below said hinge recognized by a proteolytic enzyme capable of cleaving the CH2 and/or CH3 region.

A multivalent multimer of the invention can also be produced by Immunizing a transgenic animal harboring a common variable chain with two or more antigens of interest. A panel of antibodies comprising the common variable chain and a rearranged antibody chain that specifically binds the antigen of interest is obtained from the transgenic animal. The nucleic acid encoding the common chain and the variable binding chain are then integrated into a host cells which produces an intact multivalent antibody. A multivalent multimer is then formed. Said multivalent multimer may comprise a common light chain and two or more variable binding chains, preferably wherein two of said variable binding chains are heavy chains comprising CH1, CH2 and/or CH3, which said heavy chains are paired via a hinge, typically comprised of two or more disulfide bridges between the CH1 and CH2 domain. The multivalent multimer is then cleaved with an enzyme that removes the CH2 and/or CH3 region from said heavy chains leaving at the C-termini of the heavy chains the hinge, pairing the heavy chains of the multivalent multimer, typically via two or more disulfide bonds.

Several methods have been published to favor the production of antibodies which are heterodimers. In the present invention, the cell favors the production of the heterodimers over the production of the respective homodimers. This is typically achieved by nucleic acids that encode heavy chain constant regions, preferably the CH3 region, of the heavy chains such that they favor heterodimerization (i.e. dimerization with one heavy chain combining with the second heavy chain) over homodimerization. In a preferred embodiment the multimer of the invention comprises two different immunoglobulin heavy chains with compatible heterodimerization domains.

The compatible heterodimerization domains are preferably compatible immunoglobulin heavy chain CH3 heterodimerization domains. When wildtype CH3 domains are used, co-expression of two different heavy chains (A and B) and a common light chain will result in three different antibody species, AA, AB and BB. AA and BB are designations for the two homodimer antibodies and AB is a designation for the heterodimer antibody. To increase the percentage of the desired heterodimer product (AB) CH3 engineering can be employed, or in other words, one can use heavy chains with compatible hetero-dimerization domains. The art describes various ways in which such hetero-dimerization of heavy chains can be achieved.

The term ‘compatible hetero-dimerization domains’ as used herein refers to protein domains that are engineered such that engineered domain A′ will preferentially form heterodimers with engineered domain B′ and vice versa, homo-dimerization between A′-A′ and B′-B′ is diminished.

In U.S. application Ser. No. 13/866,747 (now issued as U.S. Pat. No. 9,248,181), U.S. application Ser. No. 14/081,848 (now issued as U.S. Pat. No. 9,358,288), WO2013/157953 and WO2013/157954, methods and means are disclosed for producing multivalent antibodies using compatible heterodimerization domains. These means and methods can also be favorably employed in the present invention. Specifically, a multimer of the invention preferably comprises residues at the constant region of a first and second heavy chain to produce essentially only bispecific full length IgG molecules. Preferred residues are the amino acid L351K and T368K (EU numbering) in the first CH3 domain (wherein the first letter corresponds to the residue of the wild type CH3 domain and the second letter corresponds to the residue encoded by the CH3 that Is capable or preferentially engaging in heterodimeric pairing) or at positions corresponding thereto (the ‘KK-variant’ heavy chain) and the amino acid L351D and L388E in the second domain or at positions corresponding thereto (the ‘DE-variant’ heavy chain), or vice versa. It was previously demonstrated in our U.S. Pat. Nos. 9,248,181 and 9,358,286 as well as the WO2013/157954 PCT application that the DE-variant and KK-variant preferentially pair to form heterodimers (so-called ‘DEKK’ bispecific molecules). Homodimerization of DE-variant heavy chains (DEDE homodimers) or KK-variant heavy chains (KKKK homodimers) does not occur or does so in only negligible amounts due to repulsion between the charged residues in the CH3-CH3 interface between identical heavy chains.

In one host cell of the present invention, capable of expressing proteins that multimerize to form a multivalent multimer, the host cell is transformed with nucleic acid that encode three proteins. In order from N-terminus to C-terminus, the encoded proteins include a first protein comprising VH3-CH1---VH2-CH1-CH2-CH3, wherein a linker connects from N-terminal to C-terminal direction the CH1 to VH2 (denoted by a “---”) on the first protein, a second encoded protein comprising VLc-CL, a third encoded protein comprising VH1-CH1-CH2-CH3, wherein the CH1 domains of the first and third encoded protein pairs with the CL of the second encoded protein, and the encoded CH3 region of the first and third proteins encode amino acid L351K and T388K (EU numbering) in the first CH3 protein or at positions corresponding thereto and the amino acids L351D and L368E in the third protein or a corresponding positions thereto respectively, or vice versa. Alternatively, said first and third proteins comprise other compatible hetero-dimerization domains that cause the efficient pairing of the CH3 domains of each of these proteins.

Nucleic acids encoding said proteins can be on one or more vectors, to generate a multivalent multimer of the invention. Said nucleic acids encoding said proteins can further be stably integrated Into the host cells genome, preferably at chromosomal regions known for high expression and an absence or reduction of gene silencing.

A host cell of the present invention can be capable of producing the multivalent multimer at a purity of at least about 50%, at least about 60%, at least about 70%, at least about 80%, at least about 90%, at least about 95%, at least about 98% of the multivalent multimer of the Invention on the basis of total expressed immunoglobulin.

A host cell of the invention can be capable of producing the multivalent multimer, wherein at least about 50%, at least about 60%, least about 70%, at least about 80%, at least about 90%, at least about 95%, at least about 98% of the multivalent multimer produced comprises a variable rearranged region paired with a cognate common chain for all binding sites.

A host cell of the invention can be capable of producing the multivalent multimer, wherein at least about 50%, at least about 60%, least about 70%, at least about 80%, at least about 90%, at least about 95%, at least about 98% of the common chain expressed is paired to the multivalent multimer and is not free, unassociated protein.

Upon exposure of the multivalent multimer to a proteolytic enzyme, which cleaves said multimer below the hinge of the two dimerized heavy chains, a resulting truncated multivalent multimer or the invention is capable of being obtained, wherein at least about 50%, preferably 60%, more preferably greater than 70% and up to greater than 90% of the concentration of original protein is converted to the truncated multivalent multimer of the invention.

### Non-Human Animals

The methods and compositions described herein allow for making suitable multivalent binding proteins having binding domains obtained from, derived from, or based on suitable methods. Suitable methods can include phage display methods (including modification of germline sequences generated in phage display systems), and other in vitro methods known in the art. A particularly useful method is having a genetically modified non-human animal make, through natural processes of somatic recombination, and affinity maturation, a suitable heavy chain variable domain that can associate and express with a common light chain.

In one embodiment, the variable domains used in a multivalent multimer of the invention are obtained from, derived from or based on heavy and light chain variable regions of a non-human transgenic animal that comprises in its germline an unrearranged heavy chain variable locus and expresses a single rearranged human light chain variable domain, e.g., a common light chain mammal, such as a rodent. Such a non-human, transgenic animal upon exposure to an antigen will express a diversity of somatically rearranged heavy chain variable regions paired with a common light chain, which can then be used to develop nucleic acid sequences encoding heavy chain variable regions obtained from, derived from or based on those from said transgenic animal that are able to be efficiently transformed into host cells for the production of multivalent antibodies.

In particular, the human variable region sequences from suitable B cells of an Immunized common light chain animal that are genetically engineered to express human light chain variable domains derived from a human VL gene segment(s) can be used as a source of potential VH domains for a multivalent multimer of the invention. The B cells from said animals that are immunized with one or more antigens of interest, which are, in various embodiments, antigens to which the multivalent multimer will bind. Cells, tissues, or serum, splenic or lymph materials of the said animals are screened to obtain heavy chain variable domains (or B cells that express them) that exhibit desired characteristics with respect to the antigens of interest, e.g., high affinity, low affinity, blocking ability, activation, internalization or other characteristics. Because virtually all of the heavy chain variable domains that are generated in response to an antigenic stimulation in said transgenic animal are made in conjunction with the expressed human immunoglobulin light chain derived from preferably no more than one, or no more than two, VL gene segments, the heavy chain variable regions are capable of expressing and associating with common light chain domains that are expressed in the transgenic animal.

In one aspect, an epitope-binding protein as described herein is provided, wherein human VL and VH sequences are encoded by nucleic acid based on nucleic acid obtained from the B-cell of a transgenic mouse described herein, and/or a transgenic animal as disclosed in WO2009/157771, incorporated herein by reference, that has been immunized with an antigen comprising an epitope of Interest.

### Nucleic Acid Sequences, Polypeptides, Vectors and Cells

The invention further provides: nucleic acid sequences encoding polypeptides or linkers that can be used in the assembly of a multivalent multimer of the invention; vectors comprising such nucleic acid sequences; a cell which is capable of producing a multivalent multimer of the invention; and a method for the preparation of such a multivalent multimer using such a cell.

Multivalent antibodies according to the invention are typically produced by cells that express nucleic acid sequences encoding the polypeptides that together assemble to form a multimer of the Invention.

Accordingly, the invention provides a linker which comprises an amino acid sequence as set out in any one of SEQ ID NO: 2-25 or a polypeptide having at least about 85% sequence identity to any one thereto at least about 85% sequence identity to any one thereto, such as at least about 90% sequence identity to any one thereto, for example at least about 95% sequence identity to any one thereto, such as at least about 98% sequence identity to any one thereto, for example at least about 99% sequence identity to any one thereto.

The invention further provides a polypeptide comprising a VH3-CH1-hinge-based linker-VH2-CH1.

In certain embodiments VH3 and VH2 bind the same epitope. In certain embodiment the VH3 and VH2 bind the same antigen, but different epitopes. And in certain embodiments, VH3 and VH2 bind separate epitopes and antigens.

Also provided by the invention is a nucleic acid sequence encoding such a linker or polypeptide and a vector comprising such a nucleic acid sequence.

The nucleic acid sequences employed to make the described polypeptides can be placed in any suitable expression vector and, in appropriate circumstances, two or more vectors in a single host cell.

Generally, nucleic acid sequences encoding variable domains are cloned with the appropriate linkers and/or constant regions and the sequences are placed in operable linkage with a promoter in a suitable expression construct in a suitable cell line for expression.

### Expression of a Multivalent Multimer

Expression of antibodies in recombinant host cells has been described in the art. The nucleic acid molecules encoding the light and heavy chains of a multimer of the invention can be present as extrachromosomal copies and/or stably integrated into the chromosome of the host cell. The latter is preferred in which case a loci can be targeted that is known for lack of or reduced gene silencing.

To obtain expression of nucleic acid sequences encoding the polypeptides which assembly as a multimer of the invention, it Is well known to those skilled in the art that sequences capable of driving such expression can be functionally linked to the nucleic acid sequences encoding the polypeptides. Functionally linked is meant to describe that the nucleic acid sequences encoding the polypeptides or precursors thereof are linked to the sequences capable of driving expression such that these sequences can drive expression of the polypeptides or precursors thereof. Useful expression vectors are available in the art. e.g. the pcDNA vector series of Invitrogen. Where the sequence encoding the polypeptide of interest Is properly inserted with reference to sequences governing the transcription and translation of the encoded polypeptide, the resulting expression cassette Is useful to produce the polypeptide of interest, referred to as expression. Sequences driving expression can include promoters, enhancers and the like, and combinations thereof. These should be capable of functioning in the host cell, thereby driving expression of the nucleic acid sequences that are functionally linked to them. Promoters can be constitutive or regulated, and can be obtained from various sources, including viruses, prokaryotic, or eukaryotic sources, or artificially designed.

Expression of nucleic acid sequences of the invention can be from the natural promoter or a derivative thereof or from an entirely heterologous promoter. Some well-known and much used promoters for expression in eukaryotic cells comprise promoters derived from viruses, such as adenovirus, e.g. the E1A promoter, promoters derived from cytomegalovirus (CMV), such as the CMV immediate early (IE) promoter, promoters derived from Simian Virus 40 (SV40), and the like. Suitable promoters can also be derived from eukaryotic cells, such as metallothionein (MT) promoters, elongation factor Ia (EF-Ia) promoter, actin promoter, an immunoglobulin promoter, heat shock promoters, and the like. Any promoter or enhancer/promoter capable of driving expression of a nucleic acid sequence of the invention in a host cell is suitable in the invention. In one embodiment the sequence capable of driving expression comprises a region from a CMV promoter, preferably the region comprising nucleotides −735 to +95 of the CMV immediate early gene enhancer/promoter. The skilled person will be aware that the expression sequences used in the invention can suitably be combined with elements that can stabilize or enhance expression, such as insulators, matrix attachment regions, STAR elements and the like. This can enhance the stability and/or levels of expression.

Any cell suitable for expressing a recombinant nucleic acid sequence can be used to generate a multimer of the invention. Preferably said cell Is adapted for suspension growth.

A multivalent multimer of the invention can be expressed in host cells, typically by culturing a suitable cell of the invention and harvesting said antibody from said culture. Preferably said cell is cultured in a serum free medium. A multimer of the invention can be recovered from the cells or, preferably, from the cell culture medium by methods that are generally known to the person skilled in the art.

After recovery, an intact antibody is treated to cleave the Fc domain (e.g., CH2 and/or CH3) from the antibody. The multimers of the invention can be recovered by using methods known in the art. Such methods can include precipitation, centrifugation, filtration, size-exclusion chromatography, affinity chromatography, cation- and/or anion-exchange chromatography, hydrophobic Interaction, chromatography, and the like. Affinity chromatography, including based on the linker sequence as a means of separating the multivalent multimer of the invention can be used.

### Efficient Generation of a Truncated Multivalent Multimers

The art has employed enzymatic digestion and chemical conjugation for the production of F(ab′)2. For example, affinity vs. avidity binding of antibodies have been analyzed through the generation and comparison of an Fab to a target as compared to a F(ab′)2 examining whether a monovalent targeting causes lesser engagement with an antigen than bivalent targeting, and understanding whether bivalency leads to avidity. Prior methods of generating F(ab)2 moieties via chemical conjugation and proteolytic digestion have been unattractive for research and therapeutic applications due to inefficiencies, generation of unstable or potentially immunogenic moieties and heterogeneous mixtures of antibody fragments that make separation and use of such moieties impractical. The advent of new multivalent multimer format expressed at high purity via the techniques described herein in combination with use of enzymes capable of specific cleavage of the Fc, permit the generation of high concentrations of products of said truncated multivalent multimers by eliminating the C-terminal heterogeneity observed from pepsin digestion.

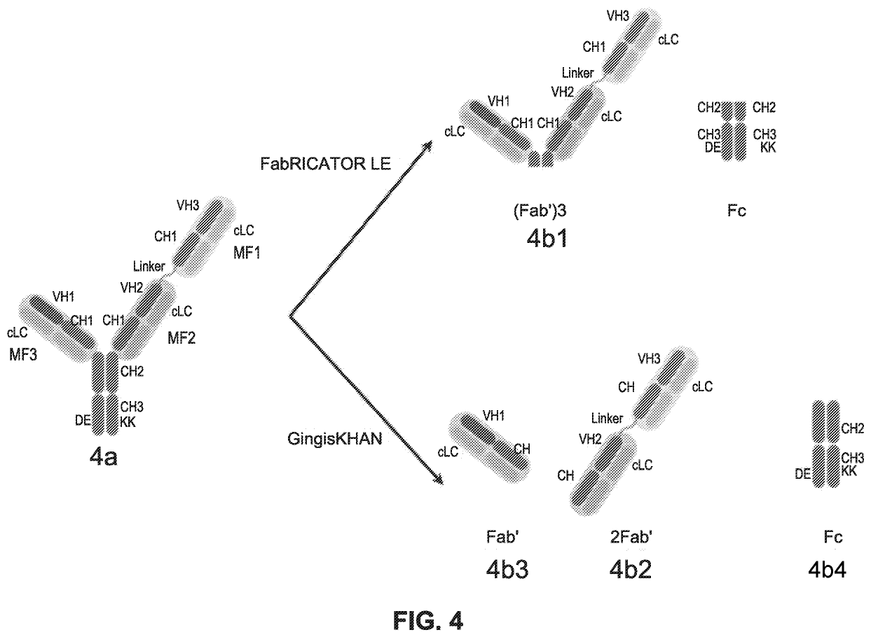

A multivalent multimer comprising F(ab′)n or (modified F(ab′)n) may be produced in connection with the methods described herein as obtained rom any full length multimer (e.g., a whole monoclonal multispecific antibody), using any suitable enzymatic cleavage and/or digestion techniques. In certain embodiments, the antibody fragment can be obtained by cleavage with the IdeS protease, an IgG-degrading enzyme of Streptococcus pyogenes that cleaves the human IgG1 at a specific site below the hinge leaving intact a F(ab′)n multimer, wherein the heavy chain on one side of the F(ab′)n is paired to the heavy chain on the other side at their respective C-terminus, wherein the pairing comprises two or more disulfide bridges. (FIG. 4b1).

Alternatively, a multivalent multimer lacking a Fc region can be obtained by use of a cysteine protease from Porphyoromonas gingivalis, that digests human IgG1 at a specific site above the hinge (KSCDK/THTCPPC) (SEQ ID NO: 92), generating intact Fab (FIG. 4b2). 2Fab′ (FIG. 4b3) and Fc (FIG. 4b4) fragments. A multimer that may be formed via this technique through the expression of a heavy chain comprising a variable domain and constant domain (e.g., CH1, CH2 and/or CH3) is connected to an additional variable domain via a linker described herein, or paired to a light chain, which is connected to an additional variable domain via a linker described herein, and wherein a proteolytic enzyme, such as from Porphyoromonas gingivalis cleaves the constant domains of said heavy chain, leaving an intact truncated 2Fab′ or multimer of more binding domains depending on how many binding domains are present on the long arm.

By use of the host cells described herein, and the heterodimerization technology, efficient generation of bispecific and multispecific moieties can be generated in large batches, which have a Fc region (e.g., CH2 and/or CH3) removed, generating a high concentration pool of F(ab′)n moeities capable of efficient research and potential therapeutic application, which provide benefits associated with a silenced (e.g., non-existent) Fc, shorter half-life, and smaller size.

### Removal of the Proteolytic Enzyme

While not required, it may be preferable to remove the proteolytic enzyme upon generation of the truncated multivalent multimers. Use of immobilized enzymes (e.g., immobilized on agarose) may also be preferable. Removal of enzymes can be accomplished through a variety of means known in the art, including the use of proteolytic enzymes that include a tag, such as a HIS-NiNTI, biotin-avidin or VSV/FLAG-anti-VSV/FLAG tag present at a terminus of the enzyme (preferably the N-terminus), permitting the enzyme to be removed via an anti-tag affinity column. The antibody fragments, such as the Fc, can also be isolated using affinity chromatography methods. Similarly, a proteolytic enzyme may be removed via charge chromatography, or a modified enzyme may be produced having an enhanced charge such that it can be removed thereafter via charge chromatography.

Alternatively, the mixture of the multivalent multimer of the invention, the proteolytic enzyme and constant domain fragment can be exposed to pH conditions or temperature conditions that are capable of denaturing the proteolytic enzyme, but not substantially interfering with the multivalent multimer pairing, thereby facilitating inactivation and separation of the enzyme, without damaging the object multivalent multimer.

### Pharmaceutical Compositions and Methods of Use

Also provided by the invention is a pharmaceutical composition which comprises a multimer of the invention and a pharmaceutically acceptable carrier and/or diluent.

Accordingly, the invention provides a multivalent multimer as described herein for use in the treatment of the human or animal body by therapy.

Further provided by the invention is a method for the treatment of a human or animal suffering from a medical condition, which method comprises administering to the human or animal a therapeutically effective amount of a multivalent multimer as described herein.

The amount of multimer according to the invention to be administered to a patient Is typically in the therapeutic window, meaning that a sufficient quantity is used for obtaining a therapeutic effect, while the amount does not exceed a threshold value leading to an unacceptable extent of side-effects. The lower the amount of multivalent multimer needed for obtaining a desired therapeutic effect, the larger the therapeutic window will typically be. A multivalent multimer according to the invention exerting sufficient therapeutic effects at low dosage is, therefore, preferred.

A reference herein to a patent document or other matter which is given as prior art is not to be taken as an admission that that document or matter was known or that the information it contains was part of the common general knowledge as at the priority date of any of the claims.

The disclosure of each reference set forth herein is incorporated herein by reference in its entirety.

### EXAMPLES

### Example 1: Comparison of Multivalent Multimer to Intact Antibody Counterpart

5 different antibodies (PG6058p02 (HER3 IgG Fc WT) SEQ ID NO: 66 & 67; PG3004p04 (HER2 IgG Fc WT) SEQ ID NO: 68 & 69; the bispecific PB11247 MF6058 (HER3)×MF3004 (HER2) Biclonics® Fc WT DEKK (US 2017/0058035) SEQ ID NO: 68-69; PG1337 (TT IgG FcWT) SEQ ID NO: 70 & 71; PB4248 (TT×TT) Biclonics® Fc WT DEKK (WO 2017/069628) SEQ ID NO: 70 & 71 were digested using the GingisKHAN Fab kit and the FabRICATOR kit (Genovis) to generate Fab and F(ab′)2 fragments, respectively. The antibody sequences are provided in Table 3.

TABLE 3

| SEQ | Composi- | |
|---|---|---|
| ID | tion | Sequence |
| 66 | DNA | ggcccagccg gccatggccc aggtgcagct |
| | encoding | ggtgcagtct ggggctgacg tgaagaagcc |
| | MF6058 | tggggcctca gtgaaggtca cgtgcaaggc |
| | | ttctggatac accttcaccg gctactatat |
| | | gcactgggtg cgacaggccc ctggacaagc |
| | | tcttgagtgg atgggatgga tcaaccctca |
| | | aagtggtggc acaaactatg caaagaagtt |
| | | tcagggcagg gtctctatga ccagggagac |
| | | gtccacaagc acagcctaca tgcagctgag |
| | | caggctgaga tctgacgaca cggctacgta |
| | | ttactgtgca agagatcatg gttctcgtca |
| | | tttctggtct tactggggct ttgattattg |
| | | gggccaaggt accctggtca cc |
| 67 | MF6058 | QVQLVQSGADVKKPGASVKVTCKASGYTFTGY |
| | | YMHWVRQAPGQALEWMGWINPQSGGTNYAKKF |
| | | QGRVSMTRETSTSTAYMQLSRLRSDDTATYYC |
| | | ARDHGSRHFWSYWGFDYWGQGTLVT |
| 68 | DNA | caggtgcagc tgaagcagtc tggggctgag |
| | encoding | ctggtgaggc ctggggcttc agtgaagctg |
| | MF3004 | tcctgcaagg cttctggcta cactttcact |
| | | ggctactata taaactgggt gaagcagagg |
| | | catggacagg gacttgagtg gattgcaagg |
| | | atttatcctg gaagtggtta tacttactac |
| | | aatgagaagt tcaagggcaa ggccacactg |
| | | actgcagaag aatcctccag cactgcctac |
| | | atgcacctca gcagcctgac atctgaggac |
| | | tctgctgtct atttctgtgc aagaccccac |
| | | tatggttacg acgactggta cttcggtgtc |
| | | tggggcacag gcaccacggt cacc |
| 69 | MF3004 | QVQLKQSGAELVRPGASVKLSCKASGYTFTGY |
| | | YINWVKQRPGQGLEWIARIYPCSGYTYYNEKF |
| | | KGKATLTAEESSSTAYMHLSSLTSEDSAVYFC |
| | | ARPHYGYDDWYFGVWGTGTTVT |
| 70 | DNA | gaggtgcagc tggtggagac tggggctgag |
| | encoding | gtgaagaagc cgggggcctc agtgaaggtc |
| | MF1337 | tcctgcaagg cttctgacta catcttcacc |
| | | aaatatgaca tcaactgggt gcgccaggcc |
| | | cctggacaag ggcttgaatg gatgggatgg |
| | | atgagcgcta acactggaaa cacgggctat |
| | | gcacagaagt tccagggcag agtcaccatg |
| | | accagggaca cgtccataaa cacagcctac |
| | | atggagctga gcagcctgac atctggtgac |
| | | acggccgttt atttctgtgc gaggagtagt |
| | | cttttcaaga cagagacggc gccctactat |
| | | cacttcgctc tggacgtctg gggccaaggg |
| | | accacggtca cc |
| 71 | MF1337 | EVQLVETGAEVKKPGASVKVSCKASDYIFTKY |
| | | DINWVRQAPGQGLEWMGWMSANTGNTGYAQKF |
| | | QGRVTMTRDTSINTAYMELSSLTSGDTAVYFC |
| | | ARSSLFKTETAPYYHFALDVWGQGTTVT |

40 and 200 units of GingisKHAN enzyme was incubated with 100 and 500 Ng/ml of HER2/HER3 multivalent antibody for 2 hours at 37° C. in the presence of 2 mM cysteine (mil reducing agent). 40 and 200 units of FabRICATOR LE was also incubated with 100 and 500 ug/ml of HER2/HER3 multivalent antibody for 2 hours at 37° C. without the addition of reducing agent. Both reactions were then purified using CaptureSelect CH1 affinity columns (Genovis). The concentrations of IgG, Fab, and F(ab′)2 were determined with Octet using Protein L sensors and are shown below in Table 4.

TABLE 4
Quantitation of antibody fragments from enzymatically digested reactions

| | PG3004p04 | PG6058p02 | PG1337p324 | PB11247p01 | PB4248p122 | Volume (μl) |
|---|---|---|---|---|---|---|
| 100 μg/mg IgG | 60.7 | 49.7 | 52.6 | 50.2 | 62.9 | 400.0 |
| 100 μg/mg IgG + 40 U | 48.2 | 38.9 | 46.2 | 47.3 | 57.8 | 400.0 |
| FabRICATOR reaction | | | | | | |
| mixture | | | | | | |
| 500 μg/mg IgG + 200 U | 220.7 | 211.6 | 219.5 | 225.3 | 221.2 | 400.0 |
| FabRICATOR reaction | | | | | | |
| mixture | | | | | | |
| 500 μg/mg IgG + 200 U | 45.6 | 44.2 | 38.9 | 32.3 | 54.8 | 550.0 |
| FabRICATOR purified | | | | | | |
| F(ab′)2 | | | | | | |
| 100 μg/mg IgG + 40 U | 64.1 | 60.9 | 57.0 | 61.5 | 66.0 | 400.0 |
| GingisKHAN reaction | | | | | | |
| mixture | | | | | | |
| 500 μg/mg IgG + 200 U | 316.1 | 319.1 | 295.1 | 312.2 | 327.5 | 400.0 |
| GingisKHAN reaction | | | | | | |
| mixture | | | | | | |
| 500 μg/mg IgG + 200 U | 87.3 | 80.1 | 64.9 | 72.0 | 73.0 | 550.0 |
| GingisKHAN purified Fab | | | | | | |

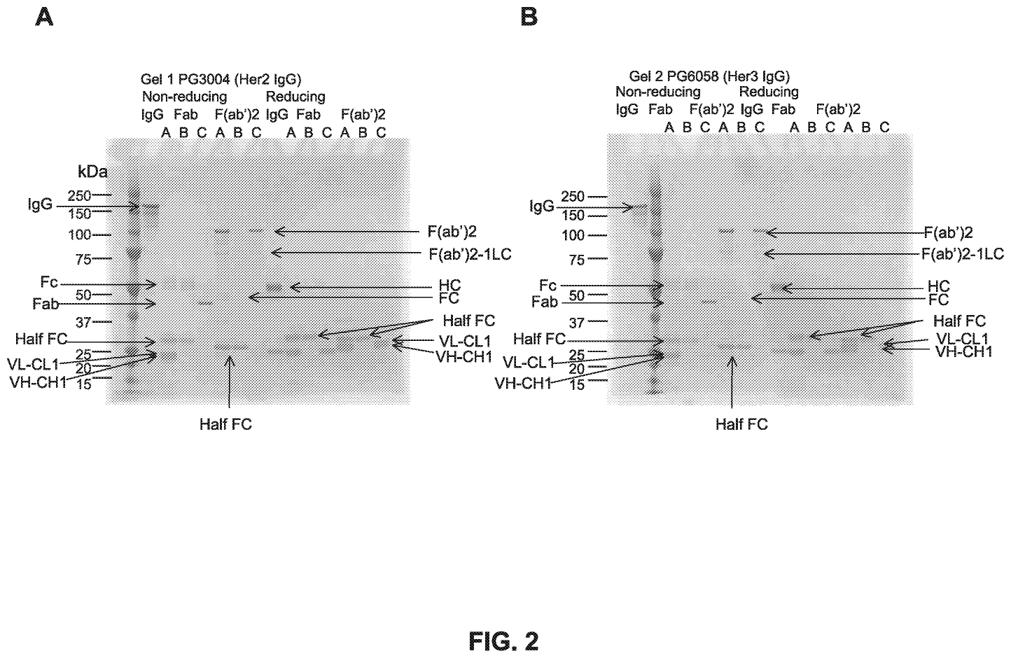

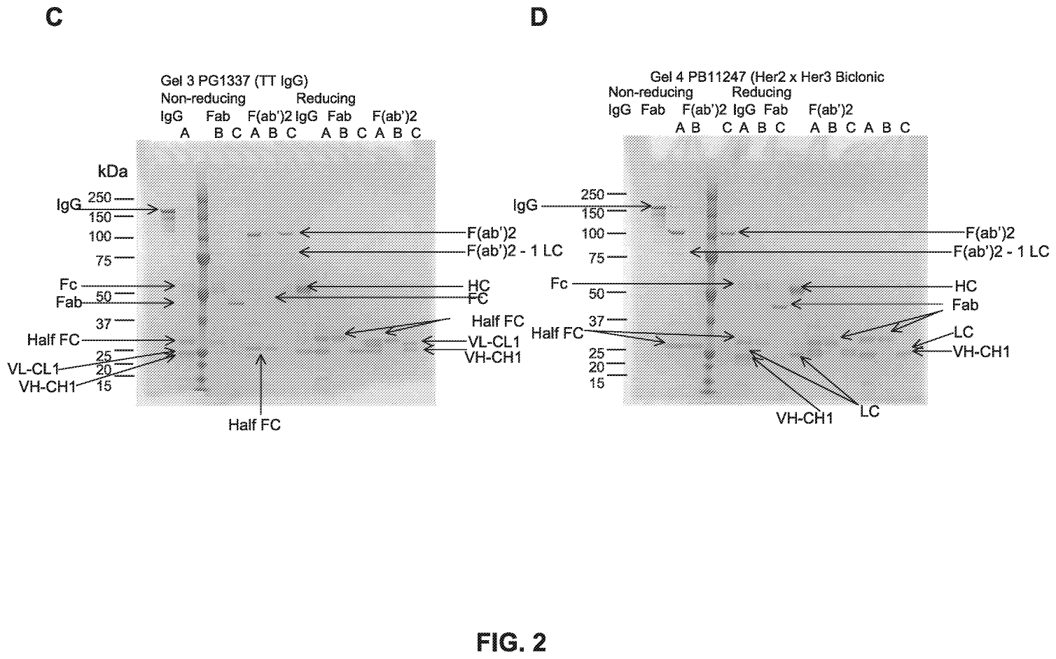

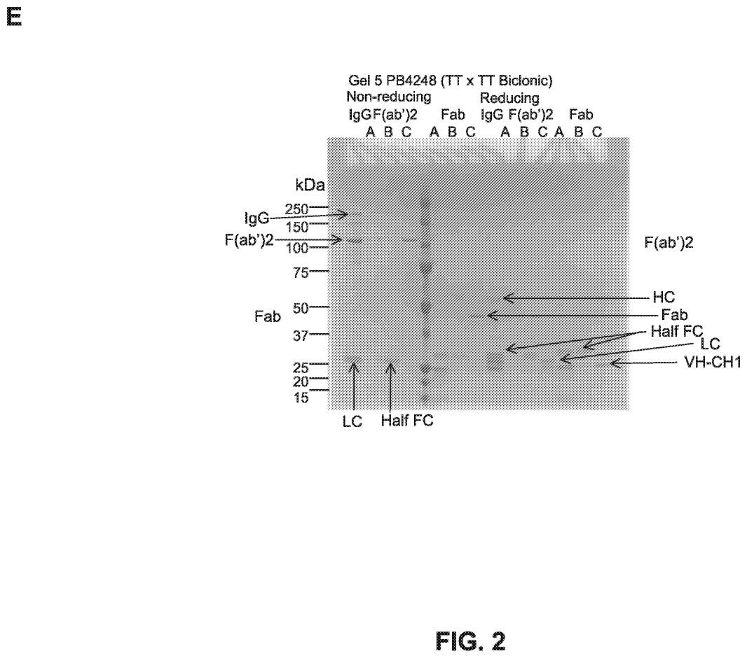

The purity was also determined by SDS-PAGE analysis as shown in FIGS. 2a-e. As shown in FIGS. 2a-e, both unpurified Fab digestions and their flow-through samples (Fc) appear partially reduced in non-reducing gels. This is likely caused by the mild reducing agent (2 mM cysteine) in the cleavage buffer. Under non-reducing conditions, the Fc in F(ab′)2 reactions is not observed. Instead, a ‘half Fc’ band appears, because the cleavage enzyme cuts below the hinge cysteine connecting the two heavy chains. Thus, non-reducing gel of ‘crude’ F(ab′)2 fragments gives the expected band sizes of 97 kDa for the F(ab′)2 and just above 25 kDa for the half Fc fragment.

The ability of intact IgG, Fab, and F(ab′)2 fragments to bind MCF7 cells was analyzed by FACS analysis. MCF7 cells were chosen because they express both HER2 and HER3.

TABLE 5
Facs binding of IgG, Fab and F(ab′)2 on MCF7 cells
Primary staining plate 1
Antibody/Fragment concentration (μg/ml)

| | | 10 | 33.3 | 1.11 | 0.37 | 0.12 | 0.041 | 0.014 | 0.0046 | 0.0015 | 0.00051 | 0.00017 | 0 |
|---|---|---|---|---|---|---|---|---|---|---|---|---|---|
| | | 1 | 2 | 3 | 4 | 5 | 6 | 7 | 8 | 9 | 10 | 11 | 12 |
| PG3004 IgG | A | 7645 | 7468 | 7585 | 7449 | 6194 | 5560 | 5192 | 5037 | 5071 | 4999 | 5038 | 4840 |
| PG6058 IgG | B | 6579 | 7623 | 7696 | 7442 | 6869 | 6023 | 5584 | 5372 | 4973 | 4925 | 4904 | 4962 |
| PG1337 IgG | C | 4492 | 4548 | 4932 | 4994 | 5070 | 5020 | 5092 | 4961 | 4974 | 4983 | 4495 | 5101 |
| PB11247 IgG | D | 8950 | 8826 | 8826 | 8721 | 7919 | 6371 | 5562 | 5312 | 5207 | 5183 | 4807 | 4891 |
| PG3004 F(ab′)2 | E | 7631 | 8129 | 8205 | 7594 | 7173 | 6008 | 5672 | 5280 | 4892 | 5011 | 4914 | 4918 |
| PG6058 F(ab′)2 | F | 5376 | 6614 | 6932 | 6859 | 7408 | 6582 | 6139 | 5310 | 5226 | 5280 | 5126 | 5090 |
| PG1337 F(ab′)2 | G | 4216 | 4715 | 4630 | 4816 | 4899 | 4894 | 4980 | 5027 | 5065 | 5209 | 4926 | 5185 |
| PB11247 F(ab′)2 | H | 9167 | 8825 | 8759 | 8902 | 8626 | 7561 | 6858 | 5987 | 5332 | 5345 | 5230 | 5197 |

Primary staining plate 2
Antibody/Fragment concentration (μg/ml)

| | | 10 | 33.3 | 1.11 | 0.37 | 0.12 | 0.041 | 0.014 | 0.0046 | 0.0015 | 0.00051 | 0.00017 | 0 |
|---|---|---|---|---|---|---|---|---|---|---|---|---|---|
| | | 1 | 2 | 3 | 4 | 5 | 6 | 7 | 8 | 9 | 10 | 11 | 12 |
| PB11247 IgG | A | 7945 | 7397 | 7704 | 6819 | 5709 | 5574 | 5039 | 4863 | 4768 | 4824 | 5044 | 5010 |
| PB4248 IgG | B | 4458 | 4637 | 4874 | 4735 | 5055 | 4941 | 4940 | 4893 | 4745 | 4723 | 4845 | 4781 |
| PB4248 F(ab′)2 | C | 4489 | 4799 | 5005 | 4699 | 4928 | 5065 | 5004 | 4783 | 4803 | 5057 | 4965 | 5059 |
| PG3004 Fab | D | 6706 | 7180 | 7500 | 6967 | 6218 | 5722 | 5160 | 5092 | 4855 | 4925 | 4973 | 5041 |
| PG6058 Fab | E | 6370 | 6749 | 6873 | 5985 | 6422 | 5910 | 5309 | 4840 | 4789 | 4872 | 4719 | 4895 |
| PG1337 Fab | F | 4157 | 4470 | 4649 | 4793 | 4613 | 4662 | 4588 | 4668 | 4745 | 4694 | 4975 | 5005 |
| PB11247 Fab | G | 7043 | 7447 | 7459 | 6870 | 6333 | 5859 | 5251 | 5151 | 5047 | 4826 | 4883 | 4886 |
| PB4248 Fab | H | 4182 | 4751 | 4605 | 4759 | 4813 | 4753 | 4784 | 4832 | 4747 | 4854 | 4748 | 4948 |

As can be seen in Table 5, the Fab and F(ab′)2 fragments generated retain their binding properties and IgG and corresponding F(ab′)2 fragments bind with similar affinity. Fab fragments also bind, although with lower affinity, as is expected. This shows that both the HER2 and HER3 arm in the F(ab)2 fragment generated using the PB11247 (MF6058 (HER3)×MF3004 (HER2)) Biclonics® are available and functional.

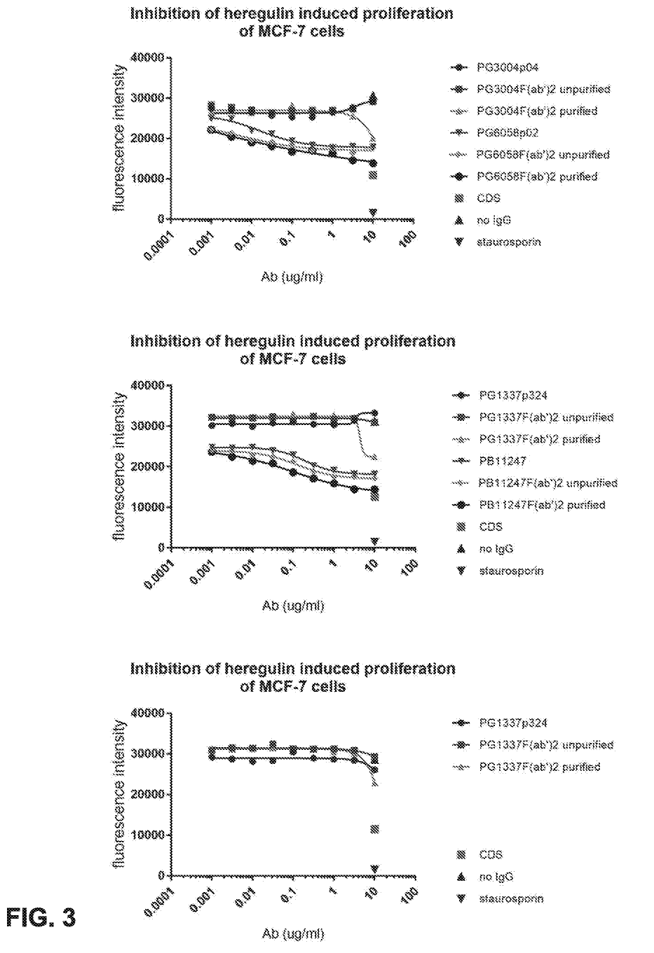

The ability of F(ab)2 fragments derived from IgG or biclonics to retain their functional activity was determined using a Heregulin-dependent MCF-7 proliferation assay. Antibodies PG3004 (Her2), PG8058 (Her3), PG1337 (TT), MF6058 (HER3)×MF3004 (HER2) Biclonics® Fc WT DEKK (US 2017/0058035) and MF1337×MF1337) (TT×TT) and the F(ab′)2 fragments produced upon exposure of the above antibodies to an enzyme capable of cleavage below the hinge (both the crude 500 μg/ml reaction mixtures and the purified F(ab′)2 fragments) were tested in a 9-point semilog titration series going down from 10 μg/ml (as measured on the Octet) as a 100% blocking control. Staurosporin (1:200) was used as a 100% blocking control. On each plate, two wells without Heregulin, two wells with Heregulin, but without inhibitor and two wells with 1:200 staurosporin (maximal Inhibition) was Included. As shown in FIG. 3, PB11247 F(ab′)2 fragments are as functional as their corresponding Biclonics® in the proliferation assay and there is no clear difference between purified or unpurified F(ab)2 activity.

### Example 2: Production of F(ab′)3 from Trivalent IgG Molecules

The ability to generate F(ab′)n fragments (including F(ab′)3, 2Fab′, and Fab fragments) from multivalent multimers was also analysed. As shown in FIG. 4, both F(ab′)3, 2Fab′ or Fab fragments are generated based on the protease chosen (e.g., FabRICATOR or GingisKHAN). The following trivalent multimers, comprising different antigen binding properties and a common light chain were analysed: PT23103p09 (having a heavy chain VH2-linker-CH1-VH3 sequence of SEQ ID NO: 72 & 73); PT23103p15 (having a heavy chain VH2-inker-CH1-VH3 sequence of SEQ ID NO: 74 & 75); PT23103p04 (having a heavy chain VH2-linker-CH1-VH3 sequence of SEQ ID NO: 76 & 77); PT23103p08 (having a heavy chain VH2-linker-CH1-VH3 sequence of SEQ ID NO: 78 & 79); PT23103p03 (having a heavy chain VH2-linker-CH1-VH3 sequence of SEQ ID NO: 80 & 81); PT23103p11 (having a heavy chain VH2-linker-CH1-VH3 sequence of SEQ ID NO: 82 & 83); and PT23103p12 (having a heavy chain VH2-inker-CH1-VH3 sequence of SEQ ID NO: 84 & 85), wherein each PT encodes the following trispecific multimer, comprising MF1337 (Tetanus toxoid) SEQ ID NO: 70 & 71 in VH3 position (top long arm), MF1122 (fibrinogen) SEQ ID NO: 88 & 87 In VH2 position (Interior long arm) and MF1025 (thyroglobulin) SEQ ID NO: 88 & 89 in VH1 position (short arm), with each VH paired with a cLC. See FIG. 4. The trivalent multimer sequences are provided in Table 6.

TABLE 6

| SEQ | | | |
|---|---|---|---|
| ID | | Composition | Sequence |
| 72 | PT23103p09 | DNA encoding | ggcccagccggccatggccgaggtgcagctggtggagactggggctg |
| | | MF1337-CH1- | aggtgaagaagccgggggcctcagtgaaggtctcctgcaaggcttct |
| | | IgG1 G4S | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggcccatcggt |
| | | | cttccccctggcaccctcctccaagtccacgtctgggggcacagcgg |
| | | | ccctgggctgcctggtcaaggactacttccccgaaccggtgacggtg |
| | | | tcgtggaactcaggcgccctgaccagcggcgtgcacaccttcccggc |
| | | | tgtcctacagtcctcaggactctactccctcagcagcgtcgtgaccg |
| | | | tgccctccagcagcttgggcacccagacctacatctgcaacgtgaat |
| | | | cacaagcccagcaacaccaaggtggacaagagagttgagcccaagag |
| | | | ctgagaaggaggtggcggcagcggcggcggaggcagcgaggtgaaga |
| | | | tggtggagtatgggggaggcgtggtccagcctgggaggtccctgaga |
| | | | ctctcctgtgcagcctctggattcaccttcagtagctatggcatgca |
| | | | ctgggtccgccaggctccaggcaaggggctggagtgggtggcagtta |
| | | | tatcatatgatggaagtaataaatactatgcagactccgtgaagggc |
| | | | cgattcaccatctccagagacaattccaagaacacgctgtatctgca |
| | | | aatgaacagcctgagagctgaggacacggccgtgtattactgtgcaa |
| | | | gagccctcttcacgaccatcgccatggactattggggccaaggtacc |
| | | | cttgtcaccgtctcgagt |
| 73 | PT23103p09 | MF1337-CH1- | AQPAMAEVQLVETGAEVKKPGASVKVSCKASDYIFTKYDINWVRQAP |
| | | IgG1 G4S | GQGLEWMGWMSANTGNTGYAQKFQGRVTMTRDTSINTAYMELSSLTS |
| | | linker-MF1122 | GDTAVYFCARSSLFKTETAPYYHFALDVWGQGTTVTVSSASTKGPSV |
| | | | PPLAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEPKS |
| | | | CDGGGGSGGGGSEVQLVESGGGVVQPGRSLRLSCAASGFTFSSYGMH |
| | | | WVRQAPGKGLEWVAVISYDGSNKYYADSVKGRFTISRDNSKNTLYLQ |
| | | | MNSLRAEDTAVYYCARALFTTIAMDYWGQGTLVTVSS |
| 74 | PT23103p15 | DNA encoding | ggcccagccggccatggccgaggtgcagctggtggagactggggctg |
| | | MF 1377-CH1- | aggtgaagaagcagggggcctcagtgaaggtctcctgcaaggcttct |
| | | IgG1H | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggcccatcggt |
| | | | cttccccctggcaccctcctccaagtccacgtctgggggcacagcgg |
| | | | ccctgggctgcctggtcaaggactacttccccgaaccggtgacggtg |
| | | | tcgtggaactcaggcgccctgaccagcggcgtgcacaccttcccggc |
| | | | tgtcctacagtcctcaggactctactccctcagcagcgtcgtgaccg |
| | | | tgccctccagcagcttgggcacccagacctacatctgcaacgtgaat |
| | | | cacaagcccagcaacaccaaggtggacaagagagttgagcccaagag |
| | | | ctgcgacaagacccacaccagcccccccagccccgctcccgagctgc |
| | | | tgggcggcgaggtgcagctggtggagtctgggggaggcgtggtccag |
| | | | cctgggaggtccatgagactctcatgtgcagcctctggattcacctt |
| | | | cagtagctatggcatgcactgggtcagccaggctccaggcaaggggc |
| | | | tggagtgggtggcagttatatcatatgatggaagtaataaatactat |
| | | | gcagactccgtgaagggccgattcaccatctccagagacaattccaa |
| | | | gaacacgctgtatctgcaaatgaacagcctgagagctgaggacacgg |
| | | | ccgtgtattactgtgcaagagccctcttcacgaccatcgccatggac |
| | | | tattggggccaaggtacccttgtcaccgtctcgagt |
| 75 | PT23103p15 | MF 1377-CH1- | AQPAMAEVQLVETGAEVKKPGASVKVSCKASDYIFTKYDINWVRQAP |
| | | IgG1 H | GQGLEWMCWMSANTGNTGYAQKFQGRVTMTRDTSINTAYMELSSLTS |
| | | linker-MF1122 | GDTAVYFCARSSLFKTETAPYYHFALDVWGQGTTVTVSSASTKGPSV |
| | | | FPLAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEPKS |
| | | | CDKTHTSPPSPAPELLGGEVQLVESGGGVVQPGRSLRLSCAASGFTF |
| | | | SSYGMHWVRQAPGKGLEWVAVISYDGSNKYYADSVKGRFTISRDNSK |
| | | | NTLYLQMNSLRAEDTAVYYCARALGTTIAMDYWGQGTLVTVSS |
| 76 | PT23103p04 | DNA encoding | ggcccagccggccatggccgaggtgcagctggtggagactggggctg |
| | | MF1377-CH1- | aggtgaagaagccgggggcctcagtgaaggtctcctgcaaggcttct |
| | | IgG2A MH | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggagccctactatcacttcgctatggacgtctggggccaa |
| | | | gggaccacggtcaccgtctccagtgctagcaccaagggccccagcgt |
| | | | gttccccctggccccctgcagccggagcaccagcgagagcaccgccg |
| | | | ccctgggctgcctggtgaaggactacttccccgagcccgtgaccgtg |
| | | | agctggaacagcggcgccctgaccagcggcgtgcacaccttccccgc |
| | | | cgtgctgcagagcagcggcctgtacagcctgagcagcgtggtgacgg |
| | | | tgcccagcagcaacttcggcacccagacctacacctgcaacgtggac |
| | | | cacaagcccagcaacaccaaggtggacaagaccgtggagcggaagag |
| | | | cagcgtggagagcccccccagccccgaggtgcagctggtggagtctg |
| | | | ggggaggcgtggtccagcctgggaggtccctgagactctcctgtgca |
| | | | gcctctggattcaccttcagtagctatggcatgcactgggtccgcca |
| | | | ggctccaggcaaggggctggagtgggtggcagttatatcatatgatg |
| | | | gaagtaataaatactatgcagactccgtgaagggccgattcaccatc |
| | | | tccagagacaattccaagaacacgctgtatctgcaaatgaacagcct |
| | | | gagagctgaggacacggccgtgtattactgtgcaagagccctcttca |
| | | | cgaccatcgccatggactattggggccaaggtacccttgtcaccgtc |
| | | | tcgagt |
| 77 | PT23103p04 | MF1377-CH1- | AQPAMAEVQLVETGAEVKKPGASVKVSCKASDYIFTKYDINWVRQAP |
| | | IgG2A MH | GQGLEWMGWMSANTGNTGYAQKFQGRVTMTRDTSINTAYMELSSLTS |
| | | linker-MF1122 | GDTAVYPCARSSLFKTETAPYYHFALDVWGQGTTVTVSSASTKGPSV |
| | | | FPLAPCSRSTSESTAALGCLVKDYFPEPVTVSWNSGALTSGVHTFPA |
| | | | VLQSSGLYSLSSVVTVPSSNFGTQTYTCNVDHKPSNTKVDKTVERKS |
| | | | SVESPPSPEVQLVESGGGVVQPGRSLRLSCAASGFTFSSYGMHWVRQ |
| | | | APGKGLHWVAVISYDGSNKYYADSVKGRFTISRDNSKNTLYLQMNSL |
| | | | RAEDTAVYYCARALFTTIAMDYWGQGTLVTVSS |
| 78 | PT23103p08 | DNA encoding | ggcccagccggccatggccgaggtgcagctggtggagactggggctg |
| | | MF1377-CH1- | aggtgaagaagccgggggcctcagtgaaggtctcctgcaaggcctct |
| | | IgG1 MH | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggcccatcggt |
| | | | cttccccctggcaccctcctccaagtccacgtctgggggcacagcgg |
| | | | ccctgggctgcctggtcaaggactacttccccgaaccggtgacggtg |
| | | | tcgtggaactcaggcgccctgaccagcggcgtgcacaccttcccggc |
| | | | tgtcctacagtcctcaggactctactccctcagcagcgtcgtgaccg |
| | | | tgccctccagcagcttgggcacccagacctacatctgcaacgtgaat |
| | | | cacaagcccagcaacaccaaggtggacaagagagttgagcccaagag |
| | | | ctgcgacaagacccacaccagcccccccagccccgaggtgcagctgg |
| | | | tggagtctgggggaggcgtggtccagcctgggaggtccctgagactc |
| | | | tcctgtgcagcctctggattcaccttcagtagctatggcatgcactg |
| | | | ggtccgccaggctccaggcaaggggctggagtgggtggcagttatat |
| | | | catatgatggaagtaataaatactatgcagactccgtgaagggccga |
| | | | ttcaccatctccagagacaattccaagaacacgctgtatctgcaaat |
| | | | gaacagcctgagagctgaggacacggccgtgtattactgtgcaagag |
| | | | ccctcttcacgaccatcgccatggactattggggccaaggtaccctt |
| | | | gtcaccgtctcgagt |
| 79 | PT23103p08 | MF1377-CH1- | AQPAMAEVQLVETGAEVKKPGASVKVSCKASDYIFTKYDINWVRQAP |
| | | IgG1 MH | GQGLEWMGWMSANTGNTGYAQKFQGRVTMTRDTSINTAYMELSSLTS |
| | | linker-MF1122 | GDTAVYFCARSSLFKTETAPYYHFALDVWGQGTTVTVSSASTKGPSV |
| | | | FPLAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEPKS |
| | | | CDKTHTSPPSPEVQLVESGGGVVQPGRSLRLSCAASGFTFSSYGMHW |
| | | | VRQAPGKGLEWVAVISYDGSNKYYADSVKGRFTISRDNSKNTLYLQM |
| | | | NSLRAEDTAVYYCARALFTTIAMDYWGQGTLVTVSS |
| 80 | PT23103p03 | DNA encoding | ggcccagccggccatggccgaggtgcagctggtggagactggggctg |
| | | MF1377-CH1- | aggtgaagaagccgggggcctcagtgaaggtctcctgcaaggcttct |
| | | IgG1 UH | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggcccatcggt |
| | | | cttccccctggcaccctcctccaagtccacgtctgggggcacagcgg |
| | | | ccctgggctgcctggtcaaggactacttccccgaaccggtgacggtg |
| | | | tcgtggaactcaggcgccctgaccagcggcgtgcacaccttccaggc |
| | | | tgtcctacagtcctcaggactctactccctcagcagcgtcgtgaccg |
| | | | tgccctccagcagcttgggcacccagacctacatctgcaacgtgaat |
| | | | cacaagcccagcaacaccaaggtggacaagagagttgagcccaagag |
| | | | ctgagacaagacccacaccgaggtgcagctggtggagtctgggggag |
| | | | gcgtggtccagcctgggaggtccctgagactctcctgtgcagcctct |
| | | | ggattcaccttcagtagctatggcatgcactgggtccgccaggctcc |
| | | | aggcaaggggctggagtgggtggcagttatatcatatgatggaagta |
| | | | ataaatactatgcagactccgtgaagggccgattcaccatctccaga |
| | | | gacaattccaagaacacgctgtatctgcaaatgaacagcctgagagc |
| | | | tgaggacacggccgtgtattactgtgcaagagccctcttcacgacca |
| | | | tcgccatggactattggggccaaggtacccttgtcaccgtctcgagt |
| 81 | PT23103p03 | MF1377-CH1- | AQPAMAEVQLVETGAEVKKPGASVKVSCKASDYIFTKYDINWVRQAP |
| | | IgG1 UH | GQGLEWMGWMSANTGNTGYAQKFQGRVTMTRDTSINTAYMELSSLTS |
| | | linker-MF1122 | GDTAVYFCARSSLFKTETAPYYHFALDVWGQGTTVTVSSASTKGPSV |
| | | | FPLAPSSKSTSGGTAALGCLVKDYFPEPVTVSWNSGALTSGVHTFPA |
| | | | VLQSSGLYSLSSVVTVPSSSLGTQTYICNVNHKPSNTKVDKRVEPKS |
| | | | CDKTHTEVQLVESGGGVVQPGRSLRLSCAASGFTFSSYGMHWVRQAP |
| | | | GKGLEWVAVISYDGSNKYYADSVKGRFTISRDNSKNTLYLQMNSLRA |
| | | | EDTAVYYCARALFTTIAMDYWGQGTLVTVSS |
| 82 | PT23103p11 | DNA encoding | Ggaccagccggccatggccgaggtgcagctggtggagactggggctg |
| | | MF1377-CH1- | aggtgaagaagcagggggcctcagtgaaggtctcctgcaaggcttct |
| | | IgG2 H | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggccccagcgt |
| | | | gttccccctggccccctgcagccggagcaccagcgagagcaccgccg |
| | | | ccctgggctgcctggtgaaggactacttccccgagcccgtgacagtg |
| | | | agctggaacagcggcgccctgaccagcggcgtgcacaccttccccgd |
| | | | cgtgctgcagagcagcggcctgtacagcctgagcagcgtggtgacgg |
| | | | tgcccagcagcaacttcggcacccagacctacacctgcaacgtggac |
| | | | cacaagcccagcaacaccaaggtggacaagaccgtggagcggaagag |
| | | | cagcgtggagagcccccccagccccgccccccccgtggccggcgagg |
| | | | tgcagctggtggagtctgggggaggcgtggtccagcctgggaggtcc |
| | | | ctgagactctcctgtgcagcctctggattcaccttcagtagctatgg |
| | | | catgcactgggtccgccaggctccaggcaaggggctggagtgggtgg |
| | | | cagttatatcatatgatggaagtaataaatactatgcagactccgtg |
| | | | aagggccgattcaccatctccagagacaattccaagaacacgctgta |
| | | | tctgcaaatgaacagcctgagagctgaggacacggccgtgtattact |
| | | | gtgcaagagccctcttcacgaccatcgccatggactattggggccaa |
| | | | ggtacccttgtcaccgtctcgagt |
| 83 | PT23103p11 | MF1377-CH1- | AQPAMAEVQLVETGAEVKKPGASVKVSCKASDYIFTKYDINWVRQAP |
| | | IgG2 H | GQGLEWMCWMSANTGNTGYAQKFQGRVTMTRDTSINTAYMELSSLTS |
| | | linker-MF1122 | GDTAVYFCARSSLFKTETAPYYHFALDVWGQGTTVTVSSASTKGPSV |
| | | | FPLAPCSRSTSESTAALGCLVKDYFPEPVTVSWNSGALTSGVHTFPA |
| | | | VLQSSGLYSLSSVVTVPSSNFGTQTYTCNVDHKPSNTKVDKTVERKS |
| | | | SVESPPSPAPPVAGEVQLVESGGGVVQPGRSLRLSCAASGFTFSSYG |
| | | | MHWVRQAPGKGLEWVAVISYDGSNKYYADSVKGRFTISRDNSKNTLY |
| | | | LQMNSLRAEDTAVYYCARALFTTIAMDYWGQGTLVTVSS |
| 84 | PT23103p12 | DNA encoding | ggcccagccggccatggccgaggtgcagctggtggagactggggctg |
| | | MF1377-CH1- | aggtgaagaagccgggggcctcagtgaaggtctcctgcaaggcttct |
| | | IgG2B H | gactacatcttcaccaaatatgacatcaactgggtgcgccaggcccc |
| | | linker-MF1122 | tggacaagggcttgaatggatgggatggatgagcgctaacactggaa |
| | | | acacgggctatgcacagaagttccagggcagagtcaccatgaccagg |
| | | | gacacgtccataaacacagcctacatggagctgagcagcctgacatc |
| | | | tggtgacacggccgtttatttctgtgcgaggagtagtcttttcaaga |
| | | | cagagacggcgccctactatcacttcgctctggacgtctggggccaa |
| | | | gggaccacggtcaccgtctccagtgctagcaccaagggccccagcgt |
| | | | gttccccctggccccctctagccggagcaccagcgagagcaccgccg |
| | | | ccctgggctgcctggtgaaggactacttccccgagcccgtgaccctg |
| | | | agctggaacagcggcgccctgaccagcggcgtgcacaccttccccgc |
| | | | cgtgctgcagagcagcggcctgtacagcctgagcagcgtggtgacgg |
| | | | tgcccagcagcaacttcggcacccagacctacacctgcaacgtggac |
| | | | cacaagcccagcaacaccaaggtggacaagaccgtggagcggaagtg |
| | | | cagcgtggagagcccccccagccccgccccccccgtggccggcgagg |
| | | | tgcagctggtggagtctgggggaggcgtggtccagcctgggaggtcc |
| | | | ctgagactctcctgtgcagcctctggattcaccttcagtagctatgg |
| | | | catgcactgggtccgccaggctccaggcaaggggctggagtgggtgg |
| | | | cagttatatcatatgatggaagtaataaatactatgcagactccgtg |
| | | | aagggccgattcaccatctccagagacaattccaagaacacgctgta |
| | | | tctgcaaatgaacagcatgagagctgaggacacggccgtgtattact |
| | | | gtgcaagagccctcttcacgaccatcgccatggactattggggccaa |
| | | | ggtacccttgtcaccgtctcgagt |
| 85 | PT23103p12 | MF1377-CH1- | AQPAMAEVQLVETGAEVKKPGASVKVSCKASDYIFTKYDINWVRQ |
| | | IgG2B H | APGQGLEWMGWMSANTGNTGYAQKFQGRVTMTRDTSINTAYMELS |
| | | linker-MF1122 | SLTSGDTAVYFCARSSLFKTETAPYYHFALDVWGQGTTVTVSSAS |
| | | | TKGPSVPPLAPSSRSTSESTAALGCLVKDYFPEPVTVSWNSGALTSG |
| | | | VHTFPAVLQSSGLYSLSSVVTVPSSNFCTQTYTCNVDHKPSNTKVDK |
| | | | TVERKCSVESPPSPAPPVAGEVQLVESGGGVVQPGRSLRLSCAASGF |
| | | | TFSSYGMEWVRQAPGKGLEWVAVISYDGSNKYYADSVKGRFTISRDN |
| | | | SKNTLYLQMNSLRAEDTAVYYCARALFTTIAMDYWGQGTLVTVSS |
| 86 | | MF1122 DNA | gaggtgcagc tggtggagtc tgggggaggc gtggtccagc |
| | | | ctgggaggtc cctgagactc tcctgtgcag cctctggatt |
| | | | caccttcagt agctatggca tgcactgggt ccgccaggct |
| | | | ccaggcaagg ggatggagtg ggtggcagtt atatcatatg |
| | | | atggaagtaa taaatactat gcagactccg tgaagggccg |
| | | | attcaccatc tccagagaca attccaagaa cacgctgtat |
| | | | ctgcaaatga acagcctgag agctgaggac acggccgtgt |
| | | | attactgtgc aagagccctc ttcacgacca tcgccatgga |
| | | | ctattggggc caaggtaccc tggtcacc |
| 87 | | MF1122 AA | EVQLVESGGGVVQPGRSLRLSCAASGFTFSSYGMHWVRQAPGKGLEW |
| | | | V |
| | | | AVISYDGSNKYYADSVKGRFTISRDNSKNTLYLQMNSLRAEDTAVYY |
| | | | CARALFTTIAMDYWGQGTLVT |
| 88 | | MF1025 DNA | gaggtgcagc tggtggagtc tgggggaggc ttggtacagc |
| | | | ctggggggtc cctgagactc tcctgtgcag cctctggatt |
| | | | cacctttagc agctatgcca tgagctgggt ccgccaggct |
| | | | ccagggaagg ggctggagtg ggtctcagct attagtggta |
| | | | gtggtggtag cacatactac gcagactccg tgaagggccg |
| | | | gttcaccatc tccagagaca attccaagaa cacgctgtat |
| | | | ctgcaaatga acagcctgag agccgaggac acggccgtgt |
| | | | attactgtgc aagggccgat tggtgggcga cttttgacta |
| | | | ctggggccaa ggtaccctgg tcacc |
| 89 | | MF1025 AA | EVQLVESGGGLVQPGGSLRLSCAASGFTFSSYAMSWVRQAPGKGLEW |
| | | | V |
| | | | SAISGSGGSTYYADSVKGRFTISRDNSKNTLYLQMNSLRAEDTAVYY |
| | | | CARADWWATFDYWGQGTLVT |

For each enzymatic reaction, 200 μg/ml IgG in a final reaction volume of 400 μl was incubated with either 80 units of FabRICATOR LE or 80 units of GingisKHAN in 10× diluted reducing buffer and was Incubated at 37° C. for 2 hours. For PT23103p09; PT23103p015; PT23103p04, 250 μl reaction mix was used for purification of the F(ab′)n using CaptureSelect CH1 columns. Flowthrough containing the Fc and the enzyme was collected as well as eluted fractions. Protein concentrations of all resulting F(ab′)n preparations were measured at A280 nm. Table 7 shows the protein concentrations for each digestion.

TABLE 7
Protein concentration of the truncated multivalent multimer (F(ab′)3) and Fab
moieties

| | | | | | Aba | Conc | Conc | Yield |
|---|---|---|---|---|---|---|---|---|
| Sample | Linker | Enzyme | Sample | Species in mixture | (280 nm) | (mg/mL) | (μM) | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | | [symbol] | [symbol] | [symbol] | [symbol] | [symbol] | [symbol] |
| | | [symbol] | [symbol] | [symbol] | [symbol] | [symbol] | [symbol] | [symbol] |
| | | | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | | [symbol] | [symbol] | [symbol] | [symbol] | [symbol] | [symbol] |
| | | [symbol] | [symbol] | [symbol] | | [symbol] | [symbol] | [symbol] |
| | | | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | | [symbol] | [symbol] | [symbol] | [symbol] | [symbol] | [symbol] |
| | | [symbol] | [symbol] | [symbol] | [symbol] | [symbol] | [symbol] | [symbol] |
| | | | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| [symbol] | [symbol] | [symbol] | [symbol] | [symbol] | [symbol] | [symbol] | [symbol] | [symbol] |
| | | [symbol] | [symbol] | [symbol] | [symbol] | [symbol] | [symbol] | [symbol] |
| | | [symbol] | [symbol] | [symbol] | [symbol] | [symbol] | [symbol] | [symbol] |

[symbol]

indicates data missing or illegible when filed

The percentage of F(ab)3 obtained in the crude extract ranged from 55-74% for PT23103p09, PT23103p15 and PT23103p04. For digestion crude extracts, the entire protein concentration was analysed against the starting material of the multivalent multimer which results in 100% of the proteins accounted for with all multivalent multimers. Percent yields are calculated by analysing the concentration (mg/ml) over molecular weight (Da) and compared to starting material of the multivalent multimer, with the molecular weight of the multivalent multimer, truncated multivalent multimer and cleaved Fc having the values shown in Table 8:

TABLE 8
Molecular weight of multimers and fragments

| | Molecular weight (kDa) |
|---|---|
| Trivalent | 195 |
| IgG/Homodimers | 146 |
| F(ab′)3 | 145 |
| half Fc | 25 |
| Fc | 51 |

To confirm specific binding of the antibody fragments, ELISA reactions to determine specific binding for tetanus, fibrinogen and thyroglobulin were undertaken. The generated truncated multivalent multimers, Fab and non-digested IgG's were tested for binding in a titration range to tetanus toxoid coated at 2 μg/ml; fibrinogen coated at 10 μg/ml; or thyroglobulin coated at 10 μg/ml. A negative control of huEGFR-Fc coated at 2.5 μg/ml was used. A protein concentration of 7.5 μg/ml for each purified moiety: F(ab′)3, Fab and 2Fab′ was analysed and PG1337p324 was included both as negative and positive control. Detection of bound F(ab)n was performed using a CH-1 detecting antibody at a 1/2000 dilution.

As shown in Table 9, all samples (non-digested, crude and purified Fabs) retained their binding to tetanus toxoid at the VH3 position, or the top position on the “long arm” of the F(ab)3 (see FIG. 4). The binding signal (Abs at 450 nm) is very similar for non-digested and digested samples.

TABLE 9
ELISA analysis of tetanus toxoid binding

| Tetanus | PG23103p09 | | | | | PT23103p15 | | | | | | | Conc. |
|---|---|---|---|---|---|---|---|---|---|---|---|---|---|
| Toxoid | | Fabricator | | Gingiskhan | | | Fabricator | | Gingiskhan | | | | Purified |
| IgG Conc | Non- | Crude | | Crude | | Non- | Crude | | Crude | | | Ctrl | sample |
| (μg/ml) | digested | mix | Elution | mix | Elution | digested | mix | Elution | mix | Elution | PG1337p324 | (—) | (μg/ml) |
| 10 | 1.031 | 0.922 | 0.959 | 1.025 | 0.987 | 0.986 | 1.029 | 1.022 | 0.941 | 0.961 | 0.979 | 0.04 | 7.5 |
| 5 | 0.974 | 0.913 | 0.909 | 0.929 | 0.899 | 0.958 | 0.933 | 0.87 | 0.873 | 0.807 | 0.814 | 0.043 | 3.75 |
| 2.5 | 0.837 | 0.956 | 0.918 | 0.896 | 0.923 | 0.936 | 0.949 | 0.922 | 0.826 | 0.852 | 0.897 | 0.042 | 1.875 |
| 1.25 | 0.785 | 0.886 | 0.863 | 0.903 | 0.887 | 0.908 | 0.941 | 0.913 | 0.844 | 0.737 | 0.909 | 0.04 | 0.9375 |
| 0.625 | 0.786 | 0.748 | 0.732 | 0.769 | 0.775 | 0.777 | 0.812 | 0.761 | 0.69 | 0.614 | 0.827 | 0.04 | 0.46875 |
| 0.3125 | 0.743 | 0.797 | 0.729 | 0.717 | 0.724 | 0.802 | 0.853 | 0.764 | 0.66 | 0.501 | 0.886 | 0.042 | 0.234675 |
| 0.15625 | 0.563 | 0.625 | 0.59 | 0.543 | 0.614 | 0.69 | 0.722 | 0.643 | 0.487 | 0.363 | 0.831 | 0.032 | 0.1171875 |
| 0.078125 | 0.413 | 0.512 | 0.437 | 0.346 | 0.451 | 0.519 | 0.573 | 0.485 | 0.337 | 0.252 | 0.765 | 0.03 | 0.05859375 |
| | | | | | | PT23103p8 | | | PT23103p3 | | | | |
| Tetanus | PG23103p04 | | | | | | Fabri- | | | Fabri- | | | Conc. |
| Toxoid | | Fabricator | | Gingiskhan | | | cator | Gingiskhan | | cator | Gingiskhan | | Purified |
| IgG Conc | Non- | Crude | | Crude | | Non- | Crude | Crude | Non- | Crude | Crude | Ctrl | sample |
| (μg/ml) | digested | mix | Elution | mix | Elution | digested | mix | mix | digested | mix | mix | (—) | (μg/ml) |
| 10 | 0.831 | 0.874 | 0.872 | 0.859 | 0.886 | 0.908 | 0.912 | 0.829 | 0.905 | 0.887 | 0.851 | 0.038 | 7.5 |
| 5 | 0.91 | 0.935 | 0.923 | 0.925 | 0.925 | .0981 | 0.958 | 0.904 | 1.014 | 0.963 | 0.916 | 0.031 | 3.75 |
| 2.5 | 0.927 | 0.971 | 0.944 | 0.914 | 0.864 | 0.922 | 0.977 | 0.874 | 1.005 | 0.982 | 0.931 | 0.03 | 1.875 |
| 1.25 | 0.869 | 0.951 | 0.899 | 0.882 | 0.863 | 0.912 | 0.991 | 0.804 | 0.954 | 0.895 | 0.836 | 0.031 | 0.9375 |
| 0.625 | 0.919 | 0.959 | 0.938 | 0.91 | 0.877 | 0.956 | 0.973 | 0.812 | 0.973 | 0.959 | 0.889 | 0.037 | 0.46875 |
| 0.3125 | 0.712 | 0.845 | 0.864 | 0.823 | 0.783 | 0.871 | 0.861 | 0.724 | 0.873 | 0.878 | 0.736 | 0.041 | 0.234675 |
| 0.15625 | 0.684 | 0.694 | 0.698 | 0.671 | 0.668 | 0.756 | 0.802 | 0.564 | 0.761 | 0.756 | 0.561 | 0.041 | 0.1171875 |
| 0.078125 | 0.512 | 0.432 | 0.514 | 0.466 | 0.457 | 0.545 | 0.576 | 0.384 | 0.549 | 0.57 | 0.377 | 0.029 | 0 |

| Tetanus | | PT23103p11 | | PT23103p12 | | | | Conc. |
|---|---|---|---|---|---|---|---|---|
| Toxoid | | Fabricator | Gingiskhan | | Fabricator | Gingiskhan | | Purified |
| IgG Conc | Non- | Crude | Crude | Non- | Crude | Crude | | sample |
| (μg/ml) | digested | mix | Mix | digested | Mix | mix | Ctrl (—) | (μg/ml) |
| 10 | 0.785 | 0.897 | 0.862 | 0.874 | 0.883 | 0.888 | 0.035 | 7.5 |
| 5 | 0.861 | 0.888 | 0.857 | 0.829 | 0.834 | 0.88 | 0.038 | 3.75 |
| 2.5 | 0.947 | 0.939 | 0.934 | 0.919 | 0.979 | 0.996 | 0.04 | 1.875 |
| 1.25 | 0.913 | 0.902 | 0.884 | 0.944 | 0.941 | 0.933 | 0.041 | 0.9375 |
| 0.625 | 0.861 | 0.926 | 0.81 | 0.862 | 0.916 | 0.824 | 0.04 | 0.46875 |
| 0.3125 | 0.644 | 0.813 | 0.709 | 0.706 | 0.83 | 0.808 | 0.036 | 0.234675 |
| 0.15625 | 0.627 | 0.686 | 0.604 | 0.559 | 0.815 | 0.643 | 0.032 | 0.1171875 |
| 0.078125 | 0.412 | 0.573 | 0.398 | 0.6964 | 0.477 | 0.438 | 0.029 | 0 |

The trivalent truncated multimers were next analysed for fibrinogen binding, which is located at the VH2 position, which Is the interior position of the long arm of the F(ab)3 (see FIG. 4). The Fabricator F(ab′)3 digest samples (non digested, crude and purified Fabs) all retained their binding to Fibrinogen (Table 10). The binding signal (Abs at 450 nm) was very similar for non-digested and digested samples. For GingisKHAN 2Fab′ (see FIG. 4), binding was retained, but was reduced for linkers IgG1G4S, IgG 2AMH, IgG 2AH and IgG 2BH. Therefore, IgGs with linkers IgG1 H, IgG1 MH and IgG1 UH all lost their binding to fibrinogen.

TABLE 10
ELISA analysis of fibrinogen binding

| | PG23103p09 | | | | | PT23103p15 | | | | | | | Conc. |
|---|---|---|---|---|---|---|---|---|---|---|---|---|---|
| Fibrinogen | | Fabricator | | Gingiskhan | | | Fabricator | | Gingiskhan | | | | Purified |
| IgG Conc | Non- | Crude | | Crude | | Non- | Crude | | Crude | | | Ctrl | sample |
| (μg/ml) | digested | mix | Elution | mix | Elution | digested | mix | Elution | mix | Elution | PG1337p324 | (—) | (μg/ml) |
| 10 | 0.793 | 0.803 | 0.779 | 0.624 | 0.628 | 0.801 | 0.779 | 0.733 | 0.08 | 0.057 | 0.039 | 0.039 | 7.5 |
| 5 | 0.738 | 0.77 | 0.728 | 0.552 | 0.564 | 0.748 | 0.781 | 0.66 | 0.065 | 0.055 | 0.04 | 0.04 | 3.75 |
| 2.5 | 0.587 | 0.644 | 0.612 | 0.367 | 0.413 | 0.709 | 0.685 | 0.599 | 0.052 | 0.049 | 0.041 | 0.039 | 1.875 |
| 1.25 | 0.382 | 0.443 | 0.367 | 0.218 | 0.26 | 0.506 | 0.442 | 0.388 | 0.047 | 0.045 | 0.046 | 0.041 | 0.9375 |
| 0.625 | 0.262 | 0.305 | 0.252 | 0.15 | 0.171 | 0.392 | 0.338 | 0.293 | 0.045 | 0.045 | 0.039 | 0.041 | 0.46875 |
| 0.3125 | 0.161 | 0.175 | 0.16 | 0.089 | 0.11 | 0.25 | 0.235 | 0.181 | 0.044 | 0.043 | 0.041 | 0.042 | 0.234675 |
| 0.15625 | 0.105 | 0.111 | 0.087 | 0.073 | 0.075 | 0.147 | 0.131 | 0.106 | 0.033 | 0.041 | 0.043 | 0.041 | 0.1171875 |
| 0 | 0.075 | 0.083 | 0.067 | 0.047 | 0.056 | 0.098 | 0.086 | 0.068 | 0.037 | 0.038 | 0.04 | 0.04 | 0 |
| | PG23103p04 | | | | | PT23103p8 | | | PT23103p3 | | | | Conc. |
| Fibrinogen | | Fabricator | | Gingiskhan | | | Fabricator | Gingiskhan | | Fabricator | Gingiskhan | | Purified |
| IgG Conc | Non- | Crude | | Crude | | Non- | Crude | Crude | Non- | Crude | Crude | Ctrl | sample |
| (μg/ml) | digested | mix | Elution | mix | Elution | digested | mix | mix | digested | mix | mix | (—) | (μg/ml) |
| 10 | 0.797 | 0.779 | 0.718 | 0.436 | 0.429 | 6.839 | 0.82 | 0.129 | 0.755 | 0.83 | 0.135 | 0.03 | 7.5 |
| 5 | 0.776 | 0.781 | 0.699 | 0.316 | 0.338 | 0.834 | 0.891 | 0.097 | 0.839 | 0.847 | 0.096 | 0.035 | 3.75 |
| 2.5 | 0.6 | 0.629 | 0.546 | 0.21 | 0.205 | 0.78 | 0.757 | 0.073 | 0.68 | 0.728 | 0.069 | 0.041 | 1.875 |
| 1.25 | 0.437 | 0.433 | 0.349 | 0.12 | 0.128 | 0.668 | 0.636 | 0.053 | 0.481 | 0.522 | 0.052 | 0.04 | 0.9375 |
| 0.625 | 0.273 | 0.288 | 0.231 | 0.086 | 0.087 | 0.482 | 0.524 | 0.065 | 0.358 | 0.365 | 0.048 | 0.041 | 0.46875 |
| 0.3125 | 0.166 | 0.174 | 0.137 | 0.069 | 0.064 | 0.291 | 0.298 | 0.045 | 0.207 | 0.22 | 0.045 | 0.039 | 0.234675 |
| 0.15625 | 0.108 | 0.112 | 0.093 | 0.056 | 0.061 | 0.188 | 0.188 | 0.044 | 0.133 | 0.137 | 0.043 | 0.04 | 0.1171875 |
| 0 | 0.073 | 0.083 | 0.066 | 0.05 | 0.045 | 0.117 | 0.115 | 0.033 | 0.084 | 0.088 | 0.036 | 0.041 | 0 |

| | PT23103p11 | | | PT23103p12 | | | | Conc. |
|---|---|---|---|---|---|---|---|---|
| Fibrinogen | | Fabricator | Gingiskhan | | Fabricator | Gingiskhan | | Purified |
| IgG Conc | Non- | Crude | Crude | Non- | Crude | Crude | | sample |
| (μg/ml) | digested | mix | Mix | digested | Mix | mix | Ctrl (—) | (μg/ml) |
| 10 | 0.775 | 0.807 | 0.334 | 6.548 | 0.707 | 0.435 | 0.04 | 7.5 |
| 5 | 0.703 | 0.686 | 0.237 | 0.443 | 0.613 | 0.311 | 0.042 | 3.75 |
| 2.5 | 0.53 | 0.525 | 0.151 | 0.305 | 0.479 | 0.207 | 0.042 | 1.875 |
| 1.25 | 0.33 | 0.34 | 0.093 | 0.221 | 0.374 | 0.162 | 0.041 | 0.9375 |
| 0.625 | 0.23 | 0.22 | 0.068 | 0.132 | 0.247 | 0.109 | 0.04 | 0.46875 |
| 0.3125 | 0.128 | 0.126 | 0.054 | 0.078 | 0.117 | 0.063 | 0.043 | 0.234675 |
| 0.15625 | 0.092 | 0.086 | 0.05 | 0.064 | 0.085 | 0.054 | 0.044 | 0.1171875 |
| 0 | 0.062 | 0.067 | 0.043 | 0.049 | 0.06 | 0.046 | 0.038 | 0 |

The trivalent multimers were also analysed for thyroglobulin binding located at the VH1, short arm position of the F(ab)3 (see FIG. 4). The FabRICATOR F(ab)3 samples (non digested, crude and purified Fabs), al retained their binding to thyroglobulin (Table 11). The binding signal (Abs at 450 nm) was very similar for non-digested and digested samples. This Fab is in VH1, short arm, position in the trivalent truncated multimer and was not affected by the enzymatic digestions. For GingisKHAN, all samples (non digested, crude and purified Fabs) retained their binding to thyroglobulin. The binding signal (Abs at 450 nm) was very similar for non-digested and digested samples.

TABLE 11
ELISA analysis of thyroglobulin binding

| Thyro- | PG23103p09 | | | | | PT23103p15 | | | | | | | Conc. |
|---|---|---|---|---|---|---|---|---|---|---|---|---|---|
| globulin | Non- | Fabricator | | Gingiskhan | | Non- | Fabricator | | Gingiskhan | | | | Purified |
| IgG Conc | di- | Crude | Elu- | Crude | | di- | Crude | | Crude | | | Ctrl | sample |
| (μg/ml) | gested | mix | tion | mix | Elution | gested | mix | Elution | mix | Elution | PG1337p324 | (—) | (μg/ml) |
| 10 | 0.911 | 0.931 | 0.905 | 0.842 | 0.824 | 0.932 | 0.95 | 0.924 | 0.855 | 0.831 | 0.041 | 0.04 | 7.5 |
| 5 | 0.879 | 0.921 | 0.889 | 0.764 | 0.813 | 0.903 | 0.958 | 0.879 | 0.798 | 0.77 | 0.044 | 0.049 | 3.75 |
| 2.5 | 0.93 | 0.976 | 0.933 | 0.873 | 0.866 | 0.998 | 0.993 | 0.986 | 0.881 | 0.815 | 0.045 | 0.039 | 1.875 |
| 1.25 | 0.865 | 0.913 | 0.905 | 0.743 | 0.814 | 0.817 | 0.882 | 0.879 | 0.777 | 0.689 | 0.047 | 0.044 | 0.9375 |
| 0.625 | 0.854 | 0.904 | 0.886 | 0.691 | 0.711 | 0.873 | 0.852 | 0.849 | 0.677 | 0.577 | 0.051 | 0.042 | 0.46875 |
| 0.3125 | 0.74 | 0.835 | 0.76 | 0.525 | 0.609 | 0.786 | 0.813 | 0.786 | 0.529 | 0.392 | 0.04 | 0.041 | 0.234675 |
| 0.15625 | 0.587 | 0.616 | 0.582 | 0.344 | 0.419 | 0.619 | 0.634 | 0.604 | 0.387 | 0.238 | 0.043 | 0.039 | 0.1171875 |
| 0 | 0.43 | 0.515 | 0.404 | 0.243 | 0.288 | 0.469 | 0.512 | 0.467 | 0.244 | 0.169 | 0.038 | 0.0639 | 0.05859375 |
| Thyro- | PG23103p04 | | | | | PT23103p8 | | | PT23103p3 | | | | Conc. |
| globulin | Non- | Fabricator | | Gingiskhan | | Non- | Fabricator | Gingiskhan | Non- | Fabricator | Gingiskhan | | Purified |
| IgG Conc | di- | Crude | Elu- | Crude | | di- | Crude | Crude | di- | Crude | Crude | Ctrl | sample |
| (μg/ml) | gested | mix | tion | mix | Elution | gested | mix | mix | gested | mix | mix | (—) | (μg/ml) |
| 10 | 0.916 | 0.939 | 0.909 | 0.833 | 0.829 | 0.928 | 0.958 | 0.89 | 0.945 | 0.943 | 0.854 | 0.038 | 7.5 |
| 5 | 0.892 | 0.943 | 0.889 | 0.806 | 0.791 | 0.85 | 0.909 | 0.774 | 0.898 | 0.886 | 0.835 | 0.04 | 3.75 |
| 2.5 | 0.88 | 0.904 | 0.866 | 0.793 | 0.774 | 0.873 | 0.92 | 0.787 | 0.897 | 0.86 | 0.826 | 0.038 | 1.875 |
| 1.25 | 0.844 | 0.813 | 80836 | 0.746 | 0.741 | 0.847 | 0.87 | 0.715 | 0.868 | 0.841 | 0.78 | 0.039 | 0.9375 |
| 0.625 | 0.79 | 0.868 | 0.833 | 0.658 | 0.671 | 0.847 | 0.891 | 0.676 | 0.867 | 0.878 | 0.683 | 0.037 | 0.46875 |
| 0.3125 | 0.704 | 0.806 | 0.714 | 0.523 | 0.501 | 0.751 | 0.798 | 0.548 | 0.739 | 0.763 | 0.621 | 0.037 | 0.234675 |
| 0.15625 | 0.558 | 0.582 | 0.55 | 0.356 | 0.344 | 0.581 | 0.669 | 0.36 | 0.59 | 0.618 | 0.373 | 0.037 | 0.1171875 |
| 0 | 0.397 | 0.474 | 0.382 | 0.234 | 0.223 | 0.456 | 0.512 | 0.239 | 0.423 | 0.472 | 0.252 | 0.035 | 0 |

| Thyro- | PT23103p11 | | | PT23103p12 | | | | Conc. |
|---|---|---|---|---|---|---|---|---|
| globulin | Non- | Fabricator | Gingiskhan | | Fabricator | Gingiskhan | | Purified |
| IgG Conc | di- | Crude | Crude | Non- | Crude | Crude | | sample |
| (μg/ml) | gested | mix | Mix | digested | Mix | mix | Ctrl (—) | (μg/ml) |
| 10 | 0.637 | 0.904 | 0.873 | 0.866 | 0.866 | 0.796 | 0.045 | 7.5 |
| 5 | 0.876 | 0.904 | 0.841 | 0.886 | 0.863 | 0.82 | 0.045 | 3.75 |
| 2.5 | 0.891 | 0.917 | 0.838 | 0.836 | 0.887 | 0.854 | 0.043 | 1.875 |
| 1.25 | 0.874 | 0.879 | 0.767 | 0.832 | 0.888 | 0.801 | 0.045 | 0.9375 |
| 0.625 | 0.822 | 0.866 | 0.7 | 0.753 | 0.801 | 0.698 | 0.045 | 0.46875 |
| 0.3125 | 0.717 | 0.751 | 0.538 | 0.652 | 0.691 | 0.542 | 0.042 | 0.234675 |
| 0.15625 | 0.597 | 0.586 | 0.389 | 0.477 | 0.598 | 0.401 | 0.049 | 0.1171875 |
| 0 | 0.419 | 0.501 | 0.273 | 0.34 | 0.445 | 0.273 | 0.04 | 0 |

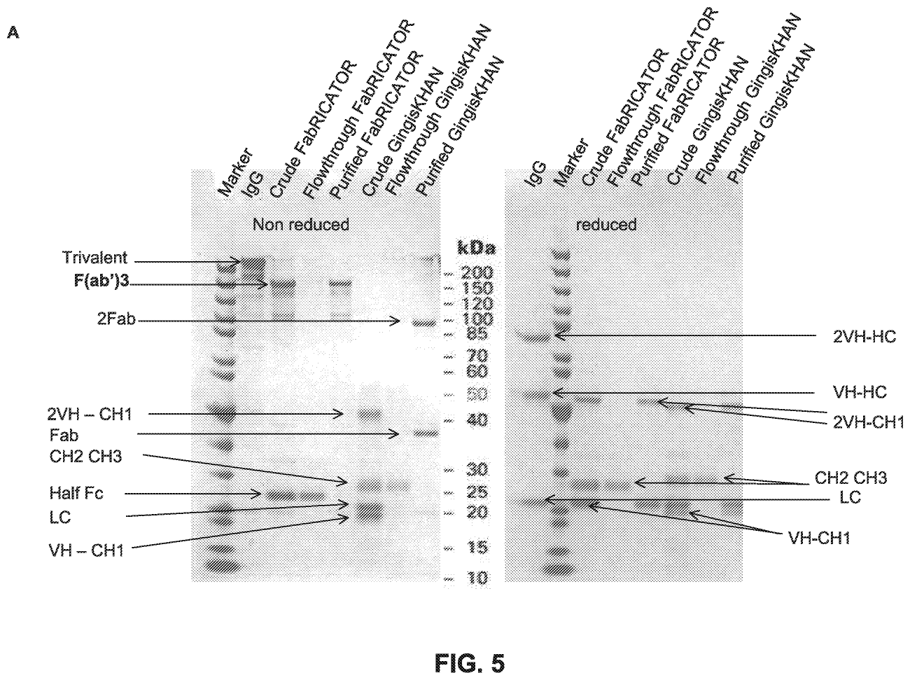

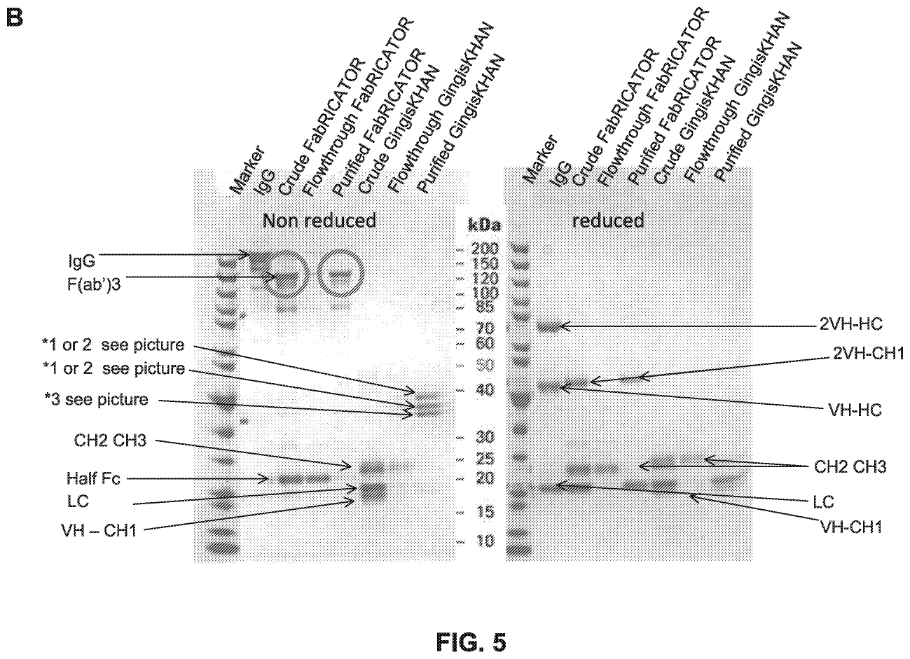

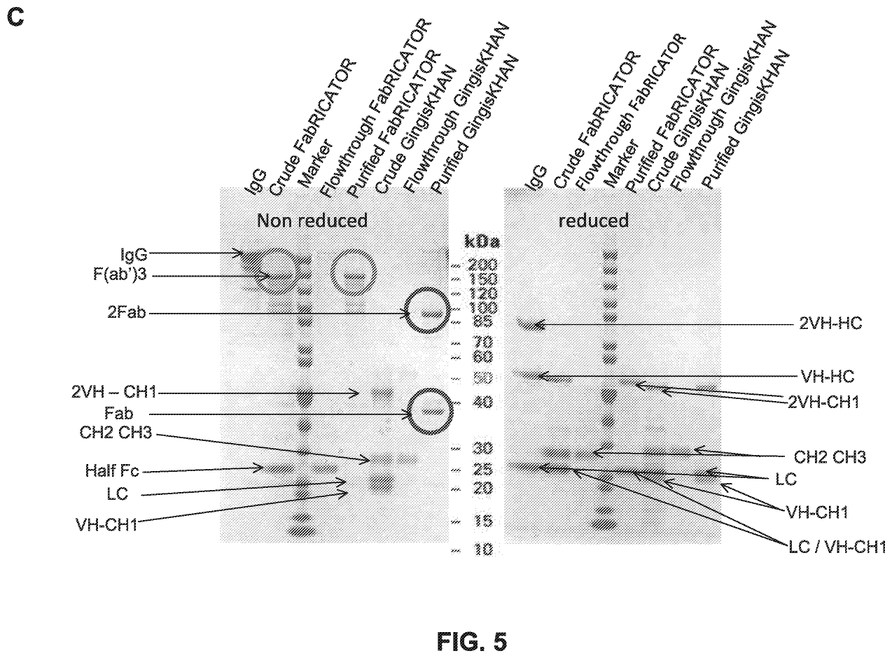

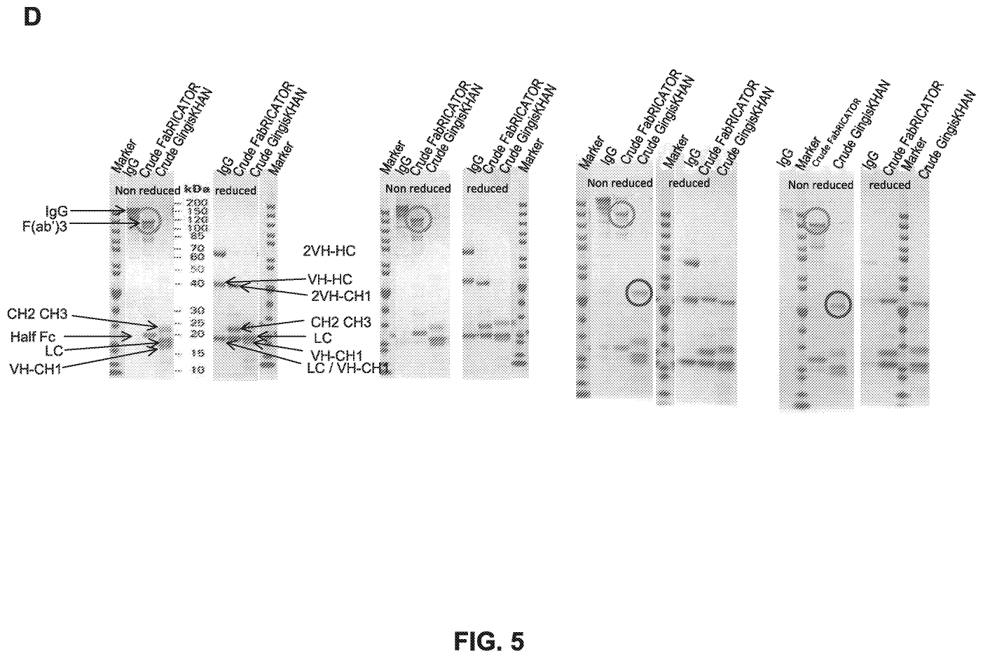

Fragment production was also confirmed by SDS-PAGE electrophoresis. As shown in FIG. 5 (a-d), Fabricator worked well for the generation of (Fab)3 fragments for each trivalent, truncated multivalent multimer. For PT23103p09, non-treated IgG (not reduced (NR) and reduced (R)) showed appropriate protein bands. Under both reducing and non-reducing conditions, the Fc in FabRICATOR reactions was not observed. Instead, a “half Fc” band is observed, because this enzyme cuts below the hinge cysteine connecting the two heavy chains resulting in two CH2-CH3 polypeptides. Both unpurified GingisKHAN reactions and its flow-through samples (Fc) appeared reduced in non-reducing gels. This was likely caused by the mild reducing agent (2 mM cysteine) as present in the cleavage buffer which results in reduction of disulphide bonds upon SDS-PAGE sample preparation. In purified GingisKHAN reactions, the expected fragments appear (FIG. 5a).

For PT23103p15, non-treated IgG (NR and R) also looked appropriate. FabRICATOR samples showed the appropriate protein bands. Under both reducing and non-reducing conditions, the Fc in FabRICATOR reactions was not observed. Instead, a “half Fc” band is observed, because this enzyme cuts below the hinge cysteine connecting the two heavy chains resulting in two CH2-CH3 polypeptides. Both unpurified GingisKHAN reactions and its flow-through samples (Fc) appeared reduced in non-reducing gels. This was likely caused by the mild reducing agent (2 mM cysteine) in the cleavage buffer which results in reduction of disulphide bonds upon SDS-PAGE sample preparation. In purified GingisKHAN reaction analysed in non-reduced SDS-PAGE, 3 bands appear around 50 kDa. These 3 bands represent the three single Fab's of the F(ab)3 and run on different heights because of parts of the linker between the two Fab's from the long arm and parts of hinges connected to these Fabs. Under reducing conditions all proteins ran at =25 kDa at the height of a VL-CL or VH-CH1 polypeptide. The binding capacity to fibrinogen in ELISA was lost in both the crude and the purified reaction, although binding capacity to tetanus and thyroglobulin in ELISA was still intact. (FIG. 5b).

For PT23103p04, non-treated IgG (NR and R) looked appropriate. All FabRICATOR samples showed the proper protein bands. Under non-reducing conditions, the Fc in FabRICATOR reactions was not observed. Instead, a “half Fc” band appeared, because this enzyme cuts below the hinge cysteine connecting the two heavy chains resulting in two CH2-CH3 polypeptides. Both unpurified GingisKHAN reactions and its flow-through samples (Fc) appeared reduced in non-reducing gels. This was likely caused by the mild reducing agent (2 mM cysteine) in the cleavage buffer which results in reduction of disulphide bonds upon SDS-PAGE sample preparation. In purified GingisKHAN reactions the expected fragments appeared (FIG. 5c).

For PT23103p08, PT23103p03, PT23103p11, and PT23103p12, non-treated IgG (NR and R) looked appropriate. All FabRICATOR samples showed the proper protein bands. Under non-reducing conditions, the Fc in FabRICATOR reactions was not observed. Instead, a “half Fc” band appeared, because this enzyme cuts below the hinge cysteine connecting the two heavy chains which results in reduction of disulphide bonds upon SDS-PAGE sample preparation. Crude GingisKHAN reactions appeared reduced in non-reducing gets. This was likely caused by the mild reducing agent (2 mM cysteine) in the cleavage buffer which results in reduction of disulphide bonds upon SDS-PAGE sample preparation. For PT23103p08 and PT23103p03 samples, FabRICATOR reactions show F(ab)3 as expected; while GingisKHAN reactions showed no 2Fab′ fragment, instead smaller bands corresponding roughly to a single Fab fragment were identified. For PT23103p11 and PT23103p12 digested with FabRICATOR generates the F(ab′)3. For PT23103p11 and PT23103p12 digested GingisKHAN generates a 2Fab′ fragment as expected although certain components are reduced when digested with GingisKHAN (FIG. 5d). It is understood that reduction could readily be mitigated by adjusting the time or reagents used.

In summary, for FabRICATOR-digested reactions al samples showed the expected protein bands. Under non-reducing conditions, the Fc in FabRICATOR reactions is not observed. Instead, a “half Fc” or “CH2-CH3” band appeared, since the enzyme cuts below the cysteine bridge connecting the two heavy chains resulting in two CH2-CH3 polypeptides.

For GingisKHAN-digested reactions generating 2Fab′, the sequence (KSCDK/THTCPPC) (SEQ ID NO: 92) as present in some linkers Is recognized by GingisKHAN, and for the following trivalent molecules the linker sequence is similar.

| (SEQ ID NO: 93) | |
|---|---|
| PT23103p15 (Linker IgG1 H) | KSCDK/THTSPPS |
| (SEQ ID NO: 93) | |
| PT23103p08 (Linker IgG1 MH) | KSCDK/THTSPPS |
| (SEQ ID NO: 93) | |
| PT23103p03 (Linker IgG1 UH) | KSCDK/THTSPPS |

It is likely therefore, that the 2Fab′ in these samples was cut into 2 separate Fabs, which correlates with the SDS-PAGE profiles. For these samples, the free linker may hinder the binding sites, which could explain the loss of binding to fibrinogen.

In summary, FabRICATOR worked well, and the methods disclosed herein generate truncated trivalent multimers (F(ab′)3), at high concentration, with specific binding maintained, and establishing that a truncated multispecific multimer (F(ab)n) having a common light chain at each Fab, and paired via heterodimerization such as DEKK can readily be generated at high concentrations. Whereas the generation of 2Fab′ fragments via use of the GingisKHAN enzyme worked for linkers lacking the modified IgG1 hinge sequence KSCDK/THTSPPS (SEQ ID NO: 93).

Thus, using the teachings disclosed herein, a person of skill in the art, may produce a substantially pure truncated, multivalent (and multispecific) multimer, including by use of the FabRICATOR enzyme. Alternatively, using a multivalent multimer, where additional binding domains are connected to said multimer via a linker that lacks a motif recognized by GingisKHAN, following the teachings disclosed herein, permits a person of skill in the art to produce a mixture of a nFab′ and Fab, where n equals two or more, and the nFab′ is comprised of a heavy chain comprising a variable domain connected to one or more additional variable domain via a linker described herein, or paired to a light chain, which is connected to one or more additional variable domain via a linker described herein.

1. A multivalent multimer comprising three or more binding domains, and comprising two or more heavy chain regions having an N-terminus and a C-terminus, wherein the multimer comprises a hinge region pairing two of said heavy chain regions at the C-terminus.

2. The multivalent multimer of claim 1, comprising three or more human heavy chain variable regions where two are paired at said hinge region, wherein said pairing comprises one or more disulfide bridges.

3. The multivalent multimer of claim 1 or 2, wherein each heavy chain region comprises a CH1 domain.

4. The multivalent multimer of any one or claims 1-3, wherein two or more heavy chain regions comprise a common variable region.

5. The multivalent multimer of any one of claims 1-3, wherein said two or more heavy chain regions are paired with a human light chain region, wherein said light chain region is a common light chain.

6. The multivalent multimer of claim 5, wherein the common light chain comprises a CL domain.

7. The multivalent multimer of claim 5 or 6, wherein the common light chain comprises the sequence or SEQ ID NO: 1.

8. The multivalent multimer of any one of claims 1-7, wherein said multimer comprises three Fab domains (Fab1, Fab2, Fab3), each comprising a heavy chain comprising a variable region (VH) and a constant region (CH1) paired with a light chain comprising a variable region (VL) and a constant region (CL), wherein Fab2 and Fab3 are connected via a linker at a heavy chain variable region of Fab2 and a CH1 domain of Fab3, and wherein the Fab1 and Fab3 are paired via a hinge comprising at least two disulfide bonds present at the C-terminus of the heavy chain of Fab1 and the heavy chain of Fab3.

9. The multivalent multimer of claim 8, wherein the linker connecting Fab2 and Fab3 comprises a sequence of SEQ ID NOs: 2-25 or a polypeptide having at least about 85% identity of said SEQ ID Nos: 2-25.

10. The multivalent multimer of any one of claims 8-9, wherein the amino acid sequence of the linker comprises a naturally-occurring sequence or comprises a sequence derived from a naturally-occurring sequence.

11. The multivalent multimer of claim 10, wherein the linker comprises a middle hinge region sequence.

12. The multivalent multimer of claim 10, wherein the linker comprises an upper and a lower hinge sequence.

13. The multivalent multimer of claim 10, wherein the linker comprises a helix-forming sequence.

14. The multivalent multimer of any one of claims 1-13, wherein the variable region of the two or more heavy chain regions specifically binds a different epitope.

15. The multivalent multimer of any one of claims 1-14, wherein the multimer binds at least two different antigens.

16. A method of producing a multivalent multimer comprising:
immunizing a transgenic animal comprising a nucleic acid encoding a common light chain variable region and an unrearranged heavy chain variable region with two or more antigens;
obtaining a panel of antibodies comprising said common light chain variable region and rearranged heavy chain antibody chains that specifically bind said two or more antigens;
integrating into a host cell, a nucleic acid encoding the common light chain variable region and two or more rearranged heavy chains, which specifically bind said two or more antigens, wherein two of said rearranged heavy chains comprise a constant region comprising CH1, CH2 and/or CH3 domain capable of pairing via the formation of a disulfide bridge;
cultivating the host cell under conditions to provide for expression of an intact multivalent multimer comprising the common light chain and two or more rearranged heavy chains, wherein two of said rearranged heavy chains are paired via a disulfide bridge between the CH1 and CH2 domain of each of said two rearranged heavy chains; and
treating the intact multivalent multimer with an enzyme that cleaves the CH2 and/or CH3 region from each of the two said rearranged heavy chains, maintaining the pairing of the two said rearranged heavy chains via a disulfide bridge to form the multivalent multimer.

17. The method or claim 16, wherein said two heavy chains comprising a constant region comprising CH1, CH2 and/or CH3 domain comprise complementary modifications to promote heterodimerization.

18. The method of claim 17, wherein the modifications are in the immunoglobulin CH2 or CH3 regions.

19. The method of claim 17 or 18, wherein the complementary modifications comprise a knob into hole, electrostatic, or DEKK modifications.

20. The method of claim 19, wherein the first of said two heavy chains comprises a first CH3 domain that dimerizes with a second CH3 domain of the second of said two heavy chains, the first CH3 of which comprises an amino acid residue lysine at positions 351 and 366 or at positions corresponding thereto and the second CH3 of which comprises the amino acid residues of aspartic acid at 351 and glutamic acid at 368 or at positions corresponding thereto.

21. The method of any one of claims 18-20, wherein the common variable regions are encoded by a nucleic acid that is obtained from, derived from or based on a nucleic acid encoded by a transgenic rodent comprising a rearranged variable chain nucleic acid sequence in its germline.

22. The method of any one of claims 18-21, further comprising recovering the multivalent multimer by tagging the enzyme and removing the enzyme via an anti-tag affinity column.

23. A multivalent multimer produced or obtainable by the method of any one of claims 16-22.

24. A cell which comprises one or more nucleic acid sequences encoding polypeptides which are capable of assembly into a multivalent multimer according to any one of claims 1-15.

25. A pharmaceutical composition which comprises a multivalent multimer of any one of claims 1-15 and a pharmaceutically acceptable carrier and/or diluent.

26. A method of treating a subject suffering from a medical indication comprising administering to the subject a therapeutically effective amount of a multivalent multimer of any one of claims 1-15.

27. A multivalent multimer of any one of claims 1-15 for use in therapy.
